(19) United States
(12) Patent Application Publication
(10) Pub. No.: US 2016/0074497 A1
(43) Pub. Date: Mar. 17, 2016

(54) STAPHYLOCOCCUS LIVE CELL VACCINES

(71) Applicant: The University of Chicago, Chicago, IL (US)

(72) Inventors: Fabiana FALUGI, Chicago, IL (US); Hwan Keun KIM, Chicago, IL (US); Dominique MISSIAKAS, Chicago, IL (US); Olaf SCHNEEWIND, Chicago, IL (US)

(73) Assignee: The University of Chicago, Chicago, IL (US)

(21) Appl. No.: 14/888,873

(22) PCT Filed: May 2, 2014
(86) PCT No.: PCT/US14/36658

§ 371 Date: Nov. 3, 2015

Related U.S. Application Data
(60) Provisional application No. 61/819,406, filed on May 3, 2013.

(51) Int. Cl. A61K 39/085; C07K 14/31

(52) U.S. Cl. CPC A61K 39/085; C07K 14/31; A61K 2039/522

(57) ABSTRACT

Staphylococcus aureus protein A variants, defective in immunoglobulin binding, elicit protective immunity against staphylococcal disease. The present invention includes methods for preventing or ameliorating staphylococcal infections, particularly hospital acquired nosocomial infections. As such, the invention contemplates vaccines for use in both active and passive immunization embodiments. In certain embodiments the vaccine is an isolated recombinant staphylococcal bacteria that expresses a variant Protein A (SpA variant) comprising (a) at least one amino acid substitution that disrupts Fc binding and (b) at least a second amino acid substitution that disrupts VH3 binding variant in at least one of SpA A, B, C, D, and/or E domains.

This application claims the benefit of priority to U.S. Provisional Patent Application Ser. No. 61/819,406, filed May 3, 2013, which is hereby incorporated by reference in its entirety.

The invention was made with government support under Grant No. U54 AI057153 awarded by the National Institutes of Health, and by a National Institute of Allergy and Infectious Diseases, Infectious Diseases Branch award AI52747. The government has certain rights in the invention.

### BACKGROUND OF THE INVENTION

1. Field of the Invention

The present invention relates generally to the field of medicine. More particularly, it concerns the use of staphylococcal live cell vaccines.

2. Background

Staphylococcus aureus is an invasive pathogen that causes skin and soft tissue infections (SSTI), bacteremia, sepsis and endocarditis (Lowy, 1998; Klevens, 2007; Fridkin, 2005). In the United States, an annual mortality of more than 20,000 is attributed to S. aureus infection, exceeding deaths caused by influenza, viral hepatitis and HIV/AIDS combined (Klevens, 2008). Of particular concern are patients with recurrent SSTI, which occurs in approximately 20% of individuals with surgical and antibiotic therapy (Kallen, 2010; Daum, 2012). Recurrent infection leads to invasive S. aureus disease with bacteremia, but is not associated with the development of immunity (Kim, 2012). Although there is an urgent need for a vaccine against S. aureus, past clinical trials with either whole cell vaccines or purified subunits have failed.

S. aureus infection of mice leads to abscess formation and disseminated disease, however, similar to humans, infected animals do not develop protective immunity (Cheng, 2009; Cheng, 2011). The contributions of several virulence mechanisms for staphylococcal infection have been revealed, including blood coagulation (Cheng, 2009; Moreillon, 1995), agglutination with fibrin cables (McAdow, 2011; McDevitt, 1994), adenosine synthesis (Thammavongsa, 2009), heme-iron scavenging (Mazmanian, 2003), toxin-mediated dissemination (Bhakdi, 1991), and escape from complement activation (de Haas, 2004; Rooijakkers, 2005). These mechanisms are crucial for the establishment of disease, however they are not required for staphylococcal escape from host adaptive immune responses. Recent work implemented protein A (SpA) as a vaccine antigen (Kim, 2010), and this prompted us to investigate its contribution to staphylococcal escape from protective immune responses. SpA is anchored in the bacterial cell wall envelope and released during staphylococcal growth (Schneewind, 1995; Ton-That, 1999). Each of its five immunoglobulin binding domains (IgBDs) capture the Fcγ domains of human or mouse IgG (Forsgren, 1966; Sjodahl, 1977; Lindmark, 1983) as well as the Fab domains of VH3 clan IgG and IgM (Cary, 1999; Forsgren, 1976). Fcγ binding to SpA is thought to protect staphylococci from opsonophagocytic killing (Forsgren, 1974). Purified SpA triggers B cell superantigen activity through crosslinking of B cell receptors, which triggers proliferative supraclonal expansion and apoptotic collapse of the activated B cells (Forsgren, 1976; Goodyear, 2003).

S. aureus is also an important pathogen of live-stock, causing large scale infections in ruminants (sheep, goats, cows), poultry and pigs. Molecular epidemiological data suggest that a common pathogenic S. aureus clone associated with ruminants originated in humans. This strain adapted to its chosen niche more than 11,000 years ago, at a time when farming domesticated animals became common practice, and then diversified. Similar jumps to the new hosts occurred for other human clinical isolates, which are now appreciated as members of the CC97, CC126, CC130, CC133, CC705 (including ST151) and CC398 clades. Adaptation to the new hosts required a combination of gene loss, allelic diversification, and acquisition of mobile genetic elements, specifically elements that support the expression of unique von-Willebrand factor binding protein alleles. Nevertheless, the core genome of ruminant associated S. aureus is stable and can lead to reciprocal transmission of newly emerging clones into the human population. This type of pathogen introduction occurs on a global scale and can be associated with the transport of live-stock or the movement of people. It has led to outbreaks of S. aureus disease in countries that otherwise have very low prevalence for staphylococcal disease.

Infection of the heifer mammary gland with S. aureus, a common mastitis pathogen, is very well documented. In lactating cows prior to calving, these infections cause significant economic loss, which has been identified by the pharmaceutical industry as a target for vaccine development. Molecular epidemiological typing revealed that a single clonal complex (CC97) is responsible for 87.4% of S. aureus bovine isolates in the United States and globally. The predicted precursor of CC97 strains was S. aureus sequence type (ST) 97 and is also represented by S. aureus Newbould 305, a chronic mastitis strain isolated from an infected teat in 1957. In addition to the conservation of five out of seven genes in the MLST analysis in CC97 isolates, the remaining MLST data permit a differentiation into >100 ST types that can be used to trace the epidemiology of live-stock associated S. aureus infection in farm animals and their transmission to humans. These data revealed that ST151 and CC398 strains can also be associated with bovine mastitis. Some of these strains, for example CC398 and ST9, represent MRSA and these clones have again entered the human population.

Efforts to eliminate pre-partum infections in heifers have focused primarily on intramammary antibiotic therapy shortly before the time of calving. While antibiotic therapy can reduce the intramammary infection (IMI) rates, an economic benefit has not been uniformly demonstrated. Further, antibiotic therapy leads to the selection of MRSA clones with the risk of these isolates re-entering the human population. Future legislation in the United States may, similar to some European countries, ban the prophylactic use of antibiotics in live-stock. An obvious strategy to eliminate bovine mastitis is vaccination. Boehringer Ingelheim Veterinary Medicine offers the only commercially available vaccine, Lysignin®, a whole-cell lysed vaccine preparation from five different phage-type S. aureus strains (the company does not reveal what strains have been included) spanning the capsular types 5, 8 and 336. Vaccine is administered as intramuscular injection of 5 ml formulated vaccine using a prime-two booster protocol with 14 day and 6 month intervals. Although initially declared to reduce the incidence of bovine mastitis in a small field trial, subsequent efficacy trials failed to demonstrate a protective effect of Lysignin®. This has been acknowledged by investigators in the field. Thus, a vaccine that can effectively prevent S. aureus mastitis in heifers or lactating cows is not yet available. If such a vaccine could be developed, it may also prevent the dissemination of S. aureus in cattle as well as the re-introduction of these strains into humans.

### SUMMARY OF THE INVENTION

Protein A (SpA)(SEQ ID NO:13), a cell wall anchored surface protein of Staphylococcus aureus, provides for bacterial evasion from innate and adaptive immune responses. Protein A binds immunoglobulins at their Fc portion, interacts with the VH3 domain of B cell receptors inappropriately stimulating B cell proliferation and apotosis, binds to von Willebrand factor A1 domains to activate intracellular clotting, and also binds to the TNF Receptor-1 to contribute to the pathogenesis of staphylococcal pneumonia. Protein A captures immunoglobulin and displays toxic attributes; here the inventors demonstrate that staphylococcal bacteria expressing variant Protein A stimulate humoral immune responses that protect against staphylococcal disease.

In certain embodiments the vaccine is an isolated recombinant staphylococcal bacteria (hereafter also referred to as “SpA variant staphylococcus”) that expresses a variant Protein A (SpA variant) comprising (a) at least one amino acid substitution that disrupts Fc binding and (b) at least a second amino acid substitution that disrupts VH3 binding variant in at least one of SpA A, B, C, D, and/or E domains. In certain aspects, the SpA variant comprises or consists of the amino acid sequence that is 80, 90, 95, 98, 99, or 100% identical to the amino acid sequence of SEQ ID NO:1 In other embodiments the SpA variant comprises a segment of SpA. The SpA segment can comprise at least or at most 1, 2, 3, 4, 5 or more IgG binding domains. The IgG domains can be at least or at most 1, 2, 3, 4, 5 or more variant A, B, C, D, or E domains. In certain aspects the SpA variant comprises at least or at most 1, 2, 3, 4, 5, or more variant A domains. In a further aspect the SpA variant comprises at least or at most 1, 2, 3, 4, 5, or more variant B domains. In still a further aspect the SpA variant comprises at least or at most 1, 2, 3, 4, 5, or more variant C domains. In yet a further aspect the SpA variant comprises at least or at most 1, 2, 3, 4, 5, or more variant D domains. In certain aspects the SpA variant comprises at least or at most 1, 2, 3, 4, 5, or more variant E domains. In a further aspect the SpA variant comprises a combination of A, B, C, D, and E domains in various combinations and permutations. The combinations can include all or part of a SpA signal peptide segment, a SpA region X segment, and/or a SpA sorting signal segment. In other aspects the SpA variant does not include a SpA signal peptide segment, a SpA region X segment, and/or a SpA sorting signal segment. In certain aspects a variant A domain comprises a substitution at position(s) 7, 8, 34, and/or 35 of SEQ ID NO:4. In another aspect a variant B domain comprises a substitution at position(s) 7, 8, 34, and/or 35 of SEQ ID NO:6. In still anther aspect a variant C domain comprises a substitution at position(s) 7, 8, 34, and/or 35 of SEQ ID NO:5. In certain aspects a variant D domain comprises a substitution at position(s) 9, 10, 36, and/or 37 of SEQ ID NO:2. In a further aspect a variant E domain comprises a substitution at position(s) 6, 7, 33, and/or 34 of SEQ ID NO:3. The following publications are specifically incorporated by reference, WO 2011/005341, WO 2012/003474, and WO 2012/034077.

In certain aspects, a SpA domain D variant or its equivalent can comprise a mutation at position 9 and 36; 9 and 37; 9 and 10; 36 and 37; 10 and 36; 10 and 37; 9, 36, and 37; 10, 36, and 37, 9, 10 and 36; or 9, 10 and 37 of SEQ ID NO:2. In a further aspect, analogous mutations can be included in one or more of domains A, B, C, or E.

In further aspects, the amino acid glutamine (Q) at position 9 of SEQ ID NO:2 (or its analogous amino acid in other SpA domains) can be replaced with an alanine (A), an asparagine (N), an aspartic acid (D), a cysteine (C), a glutamic acid (E), a phenylalanine (F), a glycine (G), a histidine (H), an isoleucine (I), a lysine (K), a leucine (L), a methionine (M), a proline (P), a serine (S), a threonine (T), a valine (V), a tryptophane (W), or a tyrosine (Y). In some aspects the glutamine at position 9 can be substituted with an arginine (R). In a further aspect, the glutamine at position 9 of SEQ ID NO:2, or its equivalent, can be substituted with a lysine or a glycine. Any 1, 2, 3, 4, 5, 6, 7, 8, 9, 10, or more of the substitutions can be explicitly excluded.

In another aspect, the amino acid glutamine (Q) at position 10 of SEQ ID NO:2 (or its analogous amino acid in other SpA domains) can be replaced with an alanine (A), an asparagine (N), an aspartic acid (D), a cysteine (C), a glutamic acid (E), a phenylalanine (F), a glycine (G), a histidine (H), an isoleucine (I), a lysine (K), a leucine (L), a methionine (M), a proline (P), a serine (S), a threonine (T), a valine (V), a tryptophane (W), or a tyrosine (Y). In some aspects the glutamine at position 10 can be substituted with an arginine (R). In a further aspect, the glutamine at position 10 of SEQ ID NO:2, or its equivalent, can be substituted with a lysine or a glycine. Any 1, 2, 3, 4, 5, 6, 7, 8, 9, 10, or more of the substitutions can be explicitly excluded.

In certain aspects, the aspartic acid (D) at position 36 of SEQ ID NO:2 (or its analogous amino acid in other SpA domains) can be replaced with an alanine (A), an asparagine (N), an arginine (R), a cysteine (C), a phenylalanine (F), a glycine (G), a histidine (H), an isoleucine (I), a lysine (K), a leucine (L), a methionine (M), a proline (P), a glutamine (Q), a serine (S), a threonine (T), a valine (V), a tryptophane (W), or a tyrosine (Y). In some aspects the aspartic acid at position 36 can be substituted with a glutamic acid (E). In certain aspects, an aspartic acid at position 36 of SEQ ID NO:2, or its equivalent, can be substituted with an alanine or a serine. Any 1, 2, 3, 4, 5, 6, 7, 8, 9, 10, or more of the substitutions can be explicitly excluded.

In another aspect, the aspartic acid (D) at position 37 of SEQ ID NO:2 (or its analogous amino acid in other SpA domains) can be replaced with an alanine (A), a an asparagine (N), an arginine (R), a cysteine (C), a phenylalanine (F), a glycine (G), a histidine (H), an isoleucine (I), a lysine (K), a leucine (L), a methionine (M), a proline (P), a glutamine (Q), a serine (S), a threonine (T), a valine (V), a tryptophane (W), or a tyrosine (Y). In some aspects the aspartic acid at position 37 can be substituted with a glutamic acid (E). In certain aspects, an aspartic acid at position 37 of SEQ ID NO:2, or its equivalent, can be substituted with an alanine or a serine. Any 1, 2, 3, 4, 5, 6, 7, 8, 9, 10, or more of the substitutions can be explicitly excluded.

In a particular embodiment the amino at position 9 of SEQ ID NO:2 (or an analogous amino acid in another SpA domain) is replaced by an alanine (A), a glycine (G), an isoleucine (I), a leucine (L), a proline (P), a serine (S), or a valine (V), In certain aspects the amino acid at position 9 of SEQ ID NO:2 is replaced by a glycine. In a further aspect the amino acid at position 9 of SEQ ID NO:2 is replaced by a lysine.

In a particular embodiment the amino at position 10 of SEQ ID NO:2 (or an analogous amino acid in another SpA domain) is replaced by an alanine (A), a glycine (G), an isoleucine (I), a leucine (L), a proline (P), a serine (S), or a valine (V), In certain aspects the amino acid at position 10 of SEQ ID NO:2 is replaced by a glycine. In a further aspect the amino acid at position 10 of SEQ ID NO:2 is replaced by a lysine.

In a particular embodiment the amino at position 36 of SEQ ID NO:2 (or an analogous amino acid in another SpA domain) is replaced by an alanine (A), a glycine (G), an isoleucine (I), a leucine (L), a proline (P), a serine (S), or a valine (V), In certain aspects the amino acid at position 36 of SEQ ID NO:2 is replaced by a serine. In a further aspect the amino acid at position 36 of SEQ ID NO:2 is replaced by an alanine.

In a particular embodiment the amino at position 37 of SEQ ID NO:2 (or an analogous amino acid in another SpA domain) is replaced by an alanine (A), a glycine (G), an isoleucine (I), a leucine (L), a proline (P), a serine (S), or a valine (V), In certain aspects the amino acid at position 37 of SEQ ID NO:2 is replaced by a serine. In a further aspect the amino acid at position 37 of SEQ ID NO:2 is replaced by an alanine.

In certain aspects the SpA variant includes a substitution of (a) one or more amino acid substitution in an IgG Fc binding sub-domain of SpA domain A, B, C, D, and/or E that disrupts or decreases binding to IgG Fc, and (b) one or more amino acid substitution in a VH3 binding sub-domain of SpA domain A, B, C, D, and/or E that disrupts or decreases binding to VH3. In still further aspects the amino acid sequence of a SpA variant comprises an amino acid sequence that is at least 50%, 60%, 70%, 80%, 90%, 95%, or 100% identical, including all values and ranges there between, to the amino acid sequence of SEQ ID NOs:2-6.

In a further aspect the SpA variant includes (a) one or more amino acid substitution in an IgG Fc binding sub-domain of SpA domain D, or at a corresponding amino acid position in other IgG domains, that disrupts or decreases binding to IgG Fc, and (b) one or more amino acid substitution in a V_{H}3 binding sub-domain of SpA domain D, or at a corresponding amino acid position in other IgG domains, that disrupts or decreases binding to V_{H}3. In certain aspects amino acid residue F5, Q9, Q10, S11, F13, Y14, L17, N28, 131, and/or K35 (SEQ ID NO:2, QQNNFNKDQQSAFYEILNMPNLNEAQRNGFIQSLKDDPSQSTNVLGEAKKLNES) of the IgG Fc binding sub-domain of domain D are modified or substituted. In certain aspects amino acid residue Q26, G29, F30, S33, D36, D37, Q40, N43, and/or E47 (SEQ ID NO:2) of the V_{H}3 binding sub-domain of domain D are modified or substituted such that binding to Fc or V_{H}3 is attenuated. In further aspects corresponding modifications or substitutions can be engineered in corresponding positions of the domain A, B, C, and/or E. Corresponding positions are defined by alignment of the domain D amino acid sequence with one or more of the amino acid sequences from other IgG binding domains of SpA, for example see FIG. 2A. In certain aspects the amino acid substitution can be any of the other 20 amino acids. In a further aspect conservative amino acid substitutions can be specifically excluded from possible amino acid substitutions. In other aspects only non-conservative substitutions are included. In any event, any substitution or combination of substitutions that reduces the binding of the domain such that SpA toxicity is significantly reduced is contemplated. The significance of the reduction in binding refers to a variant that produces minimal to no toxicity when introduced into a subject and can be assessed using in vitro methods described herein.

In certain embodiments, a variant SpA comprises at least or at most 1, 2, 3, 4, 5, 6, 7, 8, 9, 10, or more variant SpA domain D peptides. In certain aspects 1, 2, 3, 4, 5, 6, 7, 8, 9, 10, 11, 12, 13, 14, 15, 16, 17, 18, or 19 or more amino acid residues of the variant SpA are substituted or modified—including but not limited to amino acids F5, Q9, Q10, S11, F13, Y14, L17, N28, 131, and/or K35 (SEQ ID NO:2) of the IgG Fc binding sub-domain of domain D and amino acid residue Q26, G29, F30, S33, D36, D37, Q40, N43, and/or E47 (SEQ ID NO:2) of the VH3 binding sub-domain of domain D. In one aspect of the invention glutamine residues at position 9 and/or 10 of SEQ ID NO:2 (or corresponding positions in other domains) are mutated. In another aspect, aspartic acid residues 36 and/or 37 of SEQ ID NO:2 (or corresponding positions in other domains) are mutated. In a further aspect, glutamine 9 and 10, and aspartic acid residues 36 and 37 are mutated. Purified non-toxigenic SpA or SpA-D mutants/variants described herein are no longer able to significantly bind (i.e., demonstrate attenuated or disrupted binding affinity) Fcγ or F(ab)2 VH3 and also do not stimulate B cell apoptosis. These non-toxigenic Protein A variants can be used as subunit vaccines and raise humoral immune responses and confer protective immunity against S. aureus challenge. Compared to wild-type full-length Protein A or the wild-type SpA-domain D, immunization with SpA-D variants resulted in an increase in Protein A specific antibody. Using a mouse model of staphylococcal challenge and abscess formation, it was observed that immunization with the non-toxigenic Protein A variants generated significant protection from staphylococcal infection and abscess formation. As virtually all S. aureus strains express Protein A, immunization of humans with the non-toxigenic Protein A variants can neutralize this virulence factor and thereby establish protective immunity. In certain aspects the protective immunity protects or ameliorates infection by drug resistant strains of Staphylococcus, such as USA300 and other MRSA strains.

Embodiments include the use of SpA variant staphylococcus in methods and compositions for the treatment or prevention of bacterial and/or staphylococcal infection. This application also provides an immunogenic composition comprising a SpA variant staphylococcus. Furthermore, the present invention provides methods and compositions that can be used to treat (e.g., limiting staphylococcal abscess formation and/or persistence in a subject) or prevent bacterial infection. In some cases, methods for stimulating an immune response involve administering to the subject an effective amount of a composition including or encoding all or part of a SpA variant staphylococcus, and in certain aspects other bacterial proteins. Other bacterial proteins include, but are not limited to (i) a secreted virulence factor, and/or a cell surface protein or peptide, or (ii) a recombinant nucleic acid molecule encoding a secreted virulence factor, and/or a cell surface protein or peptide.

In some embodiments, SpA variant staphylococcus vaccines can be used to treat or prevent staphylococcus related disease or infection in members of the Bovidae family. In other embodiments SpA variant staphylococcus vaccines can be used to treat or prevent staphylococcus related disease or infection in members of the Bovinae subfamily. In yet other embodiments, SpA variant staphylococcus vaccines can be used to treat or prevent staphylococcus related disease or infection in domestic cattle, sheep or goats. In still other embodiments, SpA variant staphylococcus vaccines can be used to treat or prevent mastitis in livestock such as cows, goats and/or sheep. In some embodiments, mastitis may be referred to as bovine mastitits. Forms and methods of treating and/or preventing mastitis in livestock, domestic cattle, including but not limited to cows, sheep and goats are described in U.S. Pat. No. 3,425,330, U.S. Pat. No. 5,198,214, U.S. Pat. No. 6,984,381, U.S. Pat. No. 4,327,082, U.S. Pat. No. 6,544,529, U.S. Pat. No. 7,429,389, U.S. Pat. No. 4,197,290, U.S. Pat. No. 4,762,712, U.S. Pat. No. 4,840,794, U.S. Pat. No. 5,679,349, U.S. Pat. No. 8,298,542, U.S. Pat. No. 5,032,522, U.S. Pat. No. 8,313,748, U.S. Pat. No. 4,849,341, U.S. Pat. No. 4,659,656, U.S. Pat. No. 5,980,908, U.S. Pat. No. 5,198,215, U.S. Pat. No. 7,204,993, U.S. Pat. No. 8,313,752, the contents of which are incorporated herein by reference.

In some embodiments, a method of making an isolated recombinant staphylococcal bacteria is provided. In some aspects, the isolated recombinant staphylococcal bacteria is a SpA variant staphylococcus bacteria. In some embodiments, the method of making an isolated recombinant staphylococcal bacteria comprises deleting or replacing a portion of the coding region of a gene in the genome of a staphylococcal bacteria. In certain embodiments, the method of making comprises a polymerase chain reaction is used to amplify a region of interest to be introduced into or replaced in the genome of the target bacterium. In some aspects two DNA sequence segments upstream and downstream of the spa gene are amplified from chromosome of S. aureus Newman with primers:ext1F(5′GGGGACCACTTTGTACAAGAAAGCTGGGTCATTTAAGAAGATTGTTTCA GATTTATG-3′) (SEQ ID NO. 7), ext1R (5′-ATTTGTAAAGTCATCATAATATAACGAATTATGTATTGCAATACTAAAATC-3′) (SEQ ID NO. 8), and ext2F (5′-CGTCGCGAACTATAATAAAAACAAACAATACACAACGATAGATATC-3′) (SEQ ID NO. 9), ext2R(5′GGGGACAAGTTTGTACAAAAAAGCAGGCAACGAACGCCTAAAGAAAT TGTCTTTGC-3′) (SEQ ID NO. 10). In other aspects, the DNA sequences of spa_{KKAA}, spa_{AA} and spa_{KK} mutants are amplified using the primers spaF (CATAATTCGTTATATTATGATGACTTTACAAATACATACAGGG) (SEQ ID NO. 11) and spaR (GTATTGTTTGTTTTTATTATAGTTCGCGACGACGTCCA) (SEQ ID NO. 12). In still other aspects, a mutant spa gene and its two flanking region are fused together by PCR reaction. In some embodiments of the method, the amplified region may be subcloned into a plasmid to facilitate recombination in the genome of the target bacterium. In some instances the plasmid is pKOR1, described in Bae, 2005. In certain embodiments the plamid is introduced via electroporation into the bacterium of interest. In other embodiments a plasmid is introduced by any method commonly used in the art, such as heat shock, chemical transformation methods or engineered viral methods. In certain aspects, the bacterium into which the recombinant plasmid is introduced is Staphylococcus aureus. In certain aspects, after introduction of the plasmid, the bacteria are temperature shifted to 42° C. to blocking replication of plasmids and promote their insertion into the chromosome. In certain aspects, growth at 30° C. is used to promote allelic replacement. In some aspects, mutations in the gene of interest, such as the spa gene, may be verified by any of the means common in the art. In some embodiments, mutations in the gene of interest are verified by DNA sequencing of PCR amplification products.

In yet other embodiments, a method of growing an isolated recombinant staphylococcal bacteria is provided. In some aspects, the isolated recombinant staphylococcal bacteria is a SpA variant staphylococcus bacteria. In some aspects, a S. aureus strain or variant are grown in tryptic soy broth or agar at 37° C. In other aspects, a S. aureus strain or variant is S. aureus Newman or USA300 LAC. In some aspects, the S. aureus strain is grown in the presence of a selection agent. In yet other embodiments the selection agent is an antibiotic. In some embodiments, spectinomycin is used at 200 μg·mL⁻¹ to select for S. aureus plasmid selection, mutant allele selection or transposon selection. In other embodiments, erythromycin is used at 20 μg·mL⁻¹ to select for S. aureus plasmid selection, mutant allele selection or transposon selection.

Additional steps of methods may include 1, 2, 3, 4, 5, 6, 7, 8, 9, 10 or more of the following: generating the Spa variant by introducing exogenous DNA into a bacteria to encode a Spa variant; growing bacteria in media; replicating bacteria in media with or without an agent that identifies or selects for recombinant bacteria; isolating recombinant bacteria, isolating recombinant bacteria from other bacteria, such as nonrecombinant bacteria or bacteria that is not staphylococcus aureus bacteria; purifying recombinant bacteria; purifying the recombinant bacteria from other proteins or from media or from other contaminants; freezing bacteria identified as recombinant; thawing recombinant bacteria; clonally expanding recombinant bacteria; sequencing a part of the genome of recombinant bacteria, assaying recombinant bacteria for the Spa variant; detecting the Spa variant; testing the recombinant bacteria for the Spa variant, and assaying for other varians in the bacteria or for loss of the Spa variant.

In some embodiments, the recombinant staphylococcal bacteria is a staphylococcus aureus strain selected from: RN9879, RN9545, RN9547, RN9549, RN9551, RN9553, RN9555, RN9557, RN9556, RN9561, RN9563, RN9567, RN9569, RN9570, RN9571, RN9572, RN9574, RN9575, RN9576, RN9582, RN9586, RN9588, RN9590, RN9591, RN9593, RN9594, RN9596, RN9598, RN9601, RN9603, RN9606, RN9608, RN9610, RN9612, RN9616, RN9618, RN9620, RN9622, RN9623, RN9669, RN9671, RN9870, RN9871, RN9881, RN9882, RN10014, RN10021, Mu50; ATCC 700699, N315, COL, RN4220/pG01, RN4220/pG0400, A960649, SA LinR #12, SA LinR #13, SA LinR #14, N/A, NCTC8325 (RN0031), NCTC8325 (RN0153), NCTC8325 (RN2442), NCTC8325 (RN2887), GC 7647, N/A, Mu50; ATCC 700699, N315, Sanger 252, Sanger 476, NCTC 8325; RN1, COL, MW2; C1999000459; USA400; 99065, VCU006, VCU089, Mu50; ATCC 700699, Mu3; ATCC 700698, HIP5827, HIP5836, SA MER, SA MER-S6, SA MER-512, SA MER-520, HIP06297; 98-489 smw, HIP06854, HIP07256, HIP07920, HIP07930; USA600; 99758, HIP08926, HIP09143, HIP09313, HIP09433, HIP09662, HIP09735, LIM 1, LIM 2, LIM 3, 99.3795.V, N/A, HIP09740, HIP09737, BR 15, BR 5, LY-1999 0620-01, LY-1999 0620-02, LY-1999 0620-03, N/A, HIP10540, HIP10267, C2000001227, IL, N/A, N/A, P1V44, 160013, HIP12864, HIP13057, HIP13036, HIP11714, HIP11983, HIP13170, HIP13419, HIP14300, HIP15178, AIS2006032, AIS2006045, 71080, AIS 080003, AIS 1000505, AIS 1001095, AID1001123, 1002434, 1202582, Cowan I; ATCC12598; NCTC8530, No. 49, No. 56, No. 66; CN49I-Staph:I33, No. 150; 12907, No. 152; 16434, No. 153; 13111, No. 167; NCTC6571, No. 208, No. 229, No. 315; 28243, No. 326; KCM 187, No. 333, No. 344; 2748, No. 348; 605E; G2, No. 359, No. 425; 5441, No. 426; 5442, No. 430; 5446, No. 437; 96, No. 536; NCTC9789; PS80, No. 611; 46, ATCC9144; NCTC6571; NCIB6571; NRRL B-314; No. 750, No. 784, No. 690; NAG9, No. 691, No. 55-1, No. 55-2, CA-126, CA-127, CA-142, CA-224, CA-248, CA-263, CA-347, CA-374, CA-401, CA-409, CA-46, CA-513, CA-548, CA-573, CA-576, CA-632, CA-655, CA-78, CO-17, CO-23, CO-34, CO-48, CO-49, CO-61, CO-65, CO-71, CO-72, CO-84, CT-110, CT-138, CT-142, CT-174, CT-178, CT-189, CT-19, CT-228, CT-58, CT-98, GA-198, GA-210, GA-256, GA-298, GA-340, GA-355, GA-356, GA-383, GA-385, GA-442, GA-51, GA-62, GA-656, GA-73, GA-92, MN-019, MN-026, MN-030, MN-040, MN-052, MN-079, MN-082, MN-094, MN-095, MN-113, NY-12, NY-141, NY-155, NY-177, NY-208, NY-216, NY-245, NY-276, NY-282, NY-313, NY-315, NY-336, NY-51, NY-54, NY-76, OR-10, OR-130, OR-131, OR-172, OR-229, OR-25, OR-274, OR-283, OR-293, OR-297, OR-327, OR-54, TN-112, TN-113, TN-116, TN-124, TN-151, TN-65, TN-67, TN-74, TN-82, TN-90, CA-629, CA-524, CA-746A, CA-774, CA-777A, CA-852A, CA-525, CA-652, CA-726A, CA-816, CA-857A, CA-672, CO-135, CO-152, CO-178, CO-185, CO-193, CT-270, CT-287, CT-296, CT-303, CT-311, CT-390, CT-434, CT-448, CT-402, CT-413, GA-824, GA-860A, GA-1030, GA-1104, GA-1169, GA-1188, GA-810, GA-481, GA-691, GA-795, GA-806, GA-1153, GA-1216, GA-733, GA-741, GA-1026, GA-1179, MD-22, MD-12, MN-183, MN-205, MN-209, MN-218, MN-220, MN-228, MN-247, MN-268, MN-292, MN-323, MN-169, MN-194, MN-217, MN-320, MN-317, NY-454, NY-494, NY-501, NY-531, NY-581, NY-604, NY-666, NY-697, NY-706, NY-754, NY-762, NY-763, NY-769, NY-786, NY-567, NY-634, NY-650, NY-665, NY-529, OR-424, OR-477, OR-506, OR-578, OR-654, OR-434, OR-485, OR-515, OR-542, OR-589, OR-601, OR-704, TN-212, TN-213, TN-245, TN-258, TN-306, TN-256, TN-277, TN-296, TN-305, TN-296, TN-305, HIP07930; USA600; 99758, MW2; C1999000459; USA400; 99065, A890259, A940441, A910669, A970675, A850375, A920222, A960562, A970704, A970230, A970656, A900507, A910565, A950211, A960197, A910469, A950319, A960254, A930472, A950085, A980101, A870192, A890511, A900476, A860325, A950206, A910371, A970627, A970698, C1998000370, C1999000193, C1999000529, HT 20020028, HT 20020030, HT 20020037, HT 20020044, HT 20020057, HT 20020058, HT 20020065, HT 20020067, HT 20020073, HT 20020075, HT 20020141, HT 20020167, HT 20020180, HT 20020204, HT 20020229, HT 20020233, HT 20020238, HT 20020252, HT 20020261, HT 20020320, HT 20020330, HT 20020331, HT 20020338, HT 20020341, HT 20020344, HT 20020345, HT 20020351, HT 20020354, HT 20020365, HT 20020371, HT 20020372, HT 20020375, HT 20020376, HT 20020381, HT 20020390, HT 20020396, HT 20020420, HT 20020436, HT 20020438, HT 20020444, HT 20020455, HT 20020470, USA100; 626, USA200; 96758, USA300-0114, USA500; 95938, USA700; 1078, USA800; 1045, FPR 3757; USA 300, USA 1000; AIS 2006061, USA 1100; HIP 12899, HIP07930; USA600; 99758, MW2; C1999000459; USA400; 99065, USA100; 626, USA200; 96758, USA300-0114, USA500; 95938, USA700; 1078, USA800; 1045, USA 1000; AIS 2006061, USA 1100; HIP 12899, NCTC8325; RN1, NCTC8325 (RN0025), NCTC8325 (RN0027), NCTC8325 (RN0450), NCTC8325 (RN0451), NCTC8325 (RN0453), NCTC8325 (RN0981), NCTC8325 (RN1389), NCTC8325 (RN3214), NCTC8325 (RN3763), NCTC8325 (RN3984), NCTC8325 (RN4220), RN4282, NCTC8325 (RN5843), NCTC8325 (RN6390B), RN6432; “Smith diffuse”, 502A (RN6607), NCTC8325 (RN6709), NCTC8325 (RN6911), WGB4316 (RN7044), RN4850, RN4850 (RN9121), 502A (RN9120), COLVA, HIP11713, Reynolds, Becker, Cowan I; ATCC12598; NCTC8530, Wood 46, FRI361, FRI472, FRI913, MN8, MNDON, MNHOCH, A900322, A980592, HT 2000 0319, HT 2000 0509, HT 2000 0328, Newbould, Newbould 305, a strain selected from among the CC97, CC126, CC130, CC133, CC705 (including ST151) and CC398 clades. In other embodiments the recombinant staphylococcal bacteria is any human, bovine, ovine or porsine staphylococcus aureus isolate. In yet other embodiments the recombinant staphylococcal bacteria is a staphylococcus aureus isolate from any mammal.

In some embodiments the bacterial variant is attenuated insofar as the bacterial variant yields a reduced bacterial load in a host compared to a bacteria without the variation/mutation.

In some embodiments, a recombinant staphylococcal bacteria is a bacteria that has been separated from other bacteria that is not the recombinant staphyloccal bacteria or is not the particular variant of interest. In other embodiments, the bacteria may be purified away from other components in solution, such as medium.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1C—Amino acid substitutions in protein A (SpA) that abrogate Staphylococcus aureus binding to the Fcγ or F(ab)₂ domains of human IgG. (A) Diagram illustrating the binding sites in each of the five immunoglobulin binding domains (IgBDs E, D, A, B, C) of protein A and the position of substitutions that affect its association with Fcγ (SpA_{KK}) or F(ab)₂ (SpA_{AA}); H1, H2 and H3 identify helices in the triple helical bundle structure of each IgBD. (B) Immunoblotting rabbit α-SpA_{KKAA} to detect SpA in the envelope of wild-type, Δspa, spa_{KKAA}, spa_{AA} or spa_{KK} mutant S. aureus Newman as well as SpA and Sbi (staphylococcal binder of immunoglobulin) in the extracellular medium of staphylococcal cultures. (C) Top panels, merged differential interference contrast (DIC) and anti-SpA fluorescence microscopy images of wild-type and mutant S. aureus. Bars indicated 10 μm. Bottom panels, flow cytometry analysis of S. aureus strains with FITC-labeled Fcγ or F(ab)₂ fragments of human IgG.

FIG. 2A-2D—Protein A binding to immunoglobulin protects staphylococci from phagocytic killing and prevents host protective antibody responses. (A) Survival of wild-type and spa mutant S. aureus injected into the blood stream of wild-type C57BL/6 or μMT mice, lacking mature B cells and immunoglobulin (n=5, mean±SEM, *P<0.05). (B) SpA_{KKAA}-specific IgG antibodies in the serum of mice (n=10) infected with wild-type and spa mutant S. aureus (mean±SEM). (C) Kaplan-Meier study comparing the survival of mice (n=10) challenged with a lethal dose of methicillin-resistant S. aureus USA300 LAC (intravenous injection of 5×10⁷ CFU) without (naïve) or with prior infection of wild-type or spa_{KKAA} mutant S. aureus. (D) IgG antibodies specific for staphylococcal protective antigens (ClfA, FnBPB, IsdB, SpA_{KKAA}, Coa or Hla) in the serum of mice (n=10) without (naïve) or with previous infection of wild-type or spa_{KKAA} mutant S. aureus.

FIG. 3A-3C—S. aureus escape from host immune surveillances requires protein A and immunoglobulin. (A) Immunoblotting reveals SpA in the envelope and in the extracellular medium of wild-type and spa_{KKAA}/sbi mutant S. aureus cultures. (B) Natural IgG and IgM antibodies specific for spa_{KKAA}/sbi mutant S. aureus in the serum of naïve mice were detected by flow cytometry. (C) Wild-type C57BL/6 or μMT mice (n=7-9) were infected with 1×10⁷ CFU wild-type, spa_{AA} or spa_{KKAA}/sbi mutant S. aureus. Animals were euthanized and necropsied 28 days following challenge and the staphylococcal load in renal tissues determined.

FIG. 4A-4B—Binding of human immunoglobulin to protein A and its variants. (A) Human IgG, its Fcγ and F(ab)₂ fragments as well as recombinant affinity purified SpA_{KK}, SpA_{AA}, SpA_{KKAA} (IgBDs E-C) and wild-type SpA (IgBDs E-C+region X) were separated on SDS-PAGE and stained with Coomassie. Ni-NTA sepharose beads were charged with SpA_{KK}, SpA_{AA}, SpA_{KKAA} or SpA and human IgG or its Fcγ and F(ab)₂ fragments loaded on the column. The eluate was analyzed by Coomassie-stained SDS-PAGE. (B) Circular dichroism spectroscopic analysis of SpA_{KK}, SpA_{AA}, SpA_{KKAA} and SpA revealed the α-helical character of protein A and its variants.

FIG. 5A-5D—S. aureus requires protein A to escape host immune surveillances. (A) Anti-coagulated mouse blood (n=3) was incubated with 5×10⁵ CFU S. aureus Newman (wild-type) and its Δspa, spa_{KK}, spa_{AA}, and spa_{KKAA} variants for 30 minutes. Staphylococcal escape from phagocytic killing was measured by enumerating colony forming units in lysed blood samples (*P<0.05). (B) Anti-coagulated mouse blood (n=3) from C57BL/6 or μMT mice was incubated with 5×10⁵ CFU wild-type or spa_{KKAA} mutant S. aureus for 30 minutes and bacterial survival measured (*P<0.05). (C) C57BL/6 or μMT mice (n=10) were infected by intravenous injection with 1×10⁷ CFU wild-type or spa_{mA} mutant S. aureus. Twenty-eight days following challenge, the serum of infected mice was examined for IgG antibodies against protein A (SpA_{KKAA}). (D) Purified SpA, SpA_{KK}, SpA_{AA}, SpA_{KKAA} emulsified with complete Freund's adjuvant were used for immunization of mice (n=10) followed by a booster with the same antigen emulsified with incomplete Freund's adjuvant. The serum of immunized mice was examined for IgG antibodies against protein A (SpA_{KKAA}) and their IgG1, IgG2a and IgG2b subclasses.

FIG. 6—SpA binds to human, mouse and guinea pig F(ab)₂, but not to rabbit and bovine F(ab)₂.

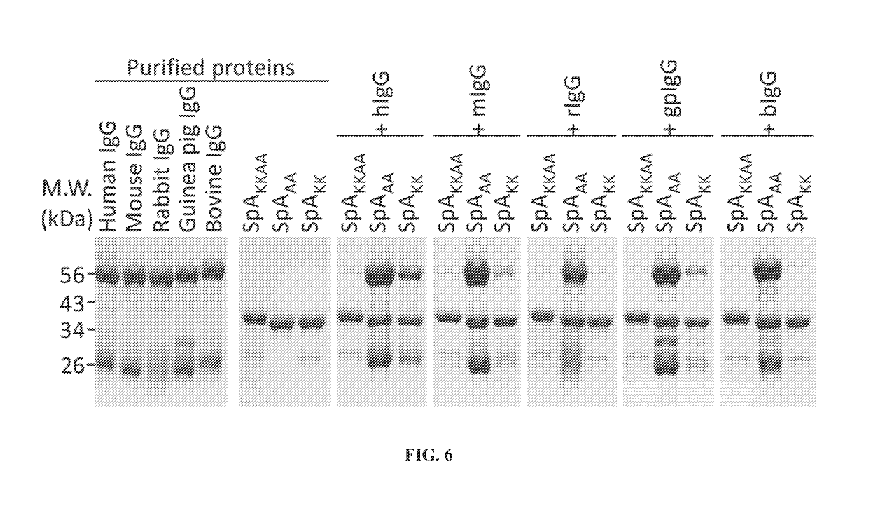

FIG. 7—Virulence defects of S. aureus spa mutants.

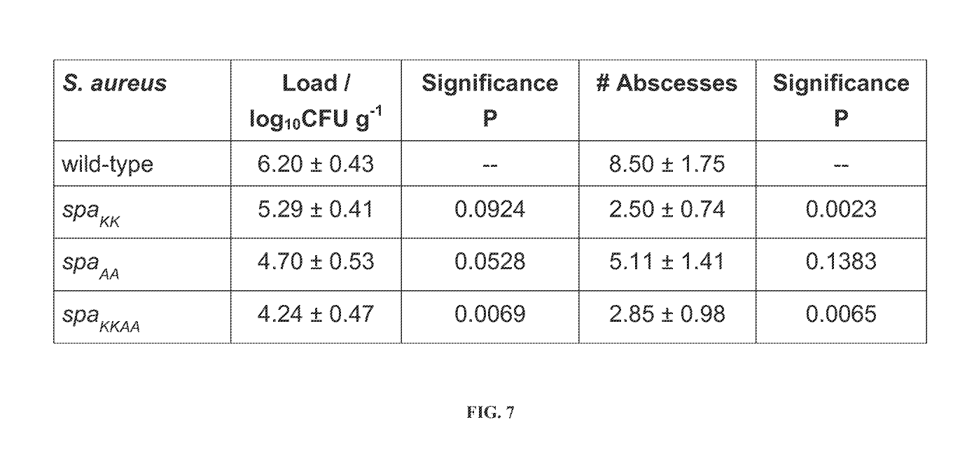

### DESCRIPTION OF ILLUSTRATIVE EMBODIMENTS

### S. Aureus Vaccines

Staphylococcus aureus is a commensal of the human skin and nares, and the leading cause of bloodstream, skin and soft tissue infections (Klevens et al., 2007). Recent dramatic increases in the mortality of staphylococcal diseases are attributed to the spread of methicillin-resistant S. aureus (MRSA) strains often not susceptible to antibiotics (Kennedy et al., 2008). In a large retrospective study, the incidence of MRSA infections was 4.6% of all hospital admissions in the United States (Klevens et al., 2007). The annual health care costs for 94,300 MRSA infected individuals in the United States exceed $2.4 billion (Klevens et al., 2007). The current MRSA epidemic has precipitated a public health crisis that needs to be addressed by development of a preventive vaccine (Boucher and Corey, 2008). To date, an FDA licensed vaccine that prevents S. aureus diseases is not available.

Previously, the inventors demonstrated that infection with virulent S. aureus Newman and clearance of the pathogen with antibiotic treatment did not aid mice in developing protective immunity against subsequent infection with the same strain. Indeed, examination of immune sera did not reveal high amounts of antibodies toward staphylococcal antigens partly due to staphylococcal protein A, a B cell superantigen. Thus, the inventors surmised that the best vaccine antigens would be encoded by genetic determinants also required for the disease process.

Here, the inventors have examined the foregoing hypothesis that staphylococcal live-attenuated vaccines can elicit protective immunity against subsequent infection with virulent S. aureus, and further, that such immunity results from antibodies against protective antigens. Mutant strains having transposon insertions in saeR, mgrA, and srtA did not persist in animal model, yet had different humoral immune response profiles. Animals infected with srtA mutant generated protective immunity against subsequent infection with the wild-type strain. Among surface molecules anchored by sortase A, AdsA and SpA were previously characterized to modulate innate and humoral immunity. Mutants with insertions into agrA, srtA, adsA and spa all had altered infectivity, but also showed altered ability to induce humoral immune response. Correlation studies between bacterial load reduction and humoral immune responses to 27 staphylococcal antigens indicated that antibodies against ClfA, FnBPB and SdrD can confer protective immunity. These and other aspects of the invention are discussed in detail below.

### I. STAPHYLOCOCCAL TARGET PROTEINS

In accordance with the present invention, altered bacteria are provided that lack the ability to express functional or “normal” versions of various proteins, as set out below. These bacteria may be engineered through a number of means, discussed further below, and may include deletion, insertion and truncation mutants in the genes in question. These altered bacteria have attenuated growth and pathogenicity, but surprisingly produce better immunity that wild-type staphylococcal strains. The following is a discussion of the relevant staphylococcal protein targets.

A. Staphylcoccal Protein A (SpA)

All Staphylococcus aureus strains express the structural gene for Protein A (spa) (Jensen, 1958; Said-Salim et al., 2003), a well characterized virulence factor whose cell wall anchored surface protein product (SpA) encompasses five highly homologous immunoglobulin binding domains designated E, D, A, B, and C (Sjodahl, 1977). These domains display ˜80% identity at the amino acid level, are 56 to 61 residues in length, and are organized as tandem repeats (Uhlen et al., 1984). SpA is synthesized as a precursor protein with an N-terminal YSIRK/GS signal peptide and a C-terminal LPXTG motif sorting signal (DeDent et al., 2008; Schneewind et al., 1992). Cell wall anchored Protein A is displayed in great abundance on the staphylococcal surface (DeDent et al., 2007; Sjoquist et al., 1972). Each of its immunoglobulin binding domains is composed of anti-parallel α-helices that assemble into a three helix bundle and bind the Fc domain of immunoglobulin G (IgG) (Deisenhofer, 1981; Deisenhofer et al., 1978), the VH3 heavy chain (Fab) of IgM (i.e., the B cell receptor) (Graille et al., 2000), the von Willebrand factor at its A1 domain [vWF AI is a ligand for platelets] (O'Seaghdha et al., 2006) and the tumor necrosis factor α (TNF-α) receptor I (TNFRI) (Gomez et al., 2006), which is displayed on surfaces of airway epithelia (Gomez et al., 2004; Gomez et al., 2007).

SpA impedes neutrophil phagocytosis of staphylococci through its attribute of binding the Fc component of IgG (Jensen, 1958; Uhlen et al., 1984). Moreover, SpA is able to activate intravascular clotting via its binding to von Willebrand factor AI domains (Hartleib et al., 2000). Plasma proteins such as fibrinogen and fibronectin act as bridges between staphylococci (CIfA and CIfB) and the platelet integrin GPIIb/IIIa (O'Brien et al., 2002), an activity that is supplemented through Protein A association with vWF AI, which allows staphylococci to capture platelets via the GPIb-α platelet receptor (Foster, 2005; O'Seaghdha et al., 2006). SpA also binds TNFRI and this interaction contributes to the pathogenesis of staphylococcal pneumonia (Gomez et al., 2004). SpA activates proinflammatory signaling through TNFR1 mediated activation of TRAF2, the p38/c-Jun kinase, mitogen activate protein kinase (MAPK) and the Rel-transcription factor NF-KB. SpA binding further induces TNFR1 shedding, an activity that appears to require the TNF-converting enzyme (TACE)(Gomez et al., 2007). All of the aforementioned SpA activities are mediated through its five IgG binding domains and can be perturbed by the same amino acid substitutions, initially defined by their requirement for the interaction between Protein A and human IgG1 (Cedergren et al., 1993.

SpA also functions as a B cell superantigen by capturing the Fab region of VH3 bearing IgM, the B cell receptor (Gomez et al., 2007; Goodyear et al., 2003; Goodyear and Silverman, 2004; Roben et al., 1995). Following intravenous challenge, staphylococcal Protein A (SpA) mutations show a reduction in staphylococcal load in organ tissues and dramatically diminished ability to form abscesses (described herein). During infection with wildtype S. aureus, abscesses are formed within forty-eight hours and are detectable by light microscopy of hematoxylin-eosin stained, thin-sectioned kidney tissue, initially marked by an influx of polymorphonuclear leukocytes (PMNs). On day 5 of infection, abscesses increase in size and enclosed a central population of staphylococci, surrounded by a layer of eosinophilic, amorphous material and a large cuff of PMNs. Histopathology revealed massive necrosis of PMNs in proximity to the staphylococcal nidus at the center of abscess lesions as well as a mantle of healthy phagocytes. The inventors also observed a rim of necrotic PMNs at the periphery of abscess lesions, bordering the eosinophilic pseudocapsule that separated healthy renal tissue from the infectious lesion. Staphylococcal variants lacking Protein A are unable to establish the histopathology features of abscesses and are cleared during infection.

In previous studies, Cedergren et al. (1993) engineered five individual substitutions in the Fc fragment binding sub-domain of the B domain of SpA, L17D, N28A, I31A and K35A. These authors created these proteins to test data gathered from a three dimensional structure of a complex between one domain of SpA and Fc₁. Cedergren et al. determined the effects of these mutations on stability and binding, but did not contemplate use of such substitutions for the production of a vaccine antigen.

Brown et al. (1998) describe studies designed to engineer new proteins based on SpA that allow the use of more favorable elution conditions when used as affinity ligands. The mutations studied included single mutations of Q13A, Q14H, N15A, N15H, F17H, Y18F, L21H, N32H, or K39H. Brown et al. report that Q13A, N15A, N15H, and N32H substitutions made little difference to the dissociation constant values and that the Y18F substitution resulted in a 2 fold decrease in binding affinity as compared to wild type SpA. Brown et al. also report that L21H and F17H substitutions decrease the binding affinity by five-fold and a hundred-fold respectively. The authors also studied analogous substitutions in two tandem domains. Thus, the Brown et al. studies were directed to generating a SpA with a more favorable elution profile, hence the use of His substitutions to provide a pH sensitive alteration in the binding affinity. Brown et al. is silent on the use of SpA as a vaccine antigen.

Graille et al. (2000) describe a crystal structure of domain D of SpA and the Fab fragment of a human IgM antibody. Graille et al. define by analysis of a crystal structure the D domain amino acid residues that interact with the Fab fragment as residues Q26, G29, F30, Q32, S33, D36, D37, Q40, N43, E47, or L51, as well as the amino acid residues that form the interface between the domain D sub-domains. Graille et al. define the molecular interactions of these two proteins, but is silent in regard to any use of substitutions in the interacting residues in producing a vaccine antigen.

O'Seaghdha et al. (2006) describe studies directed at elucidating which sub-domain of domain D binds vWF. The authors generated single mutations in either the Fc or VH3 binding sub-domains, i.e., amino acid residues F5A, Q9A, Q10A, F13A, Y14A, L17A, N28A, 131A, K35A, G29A, F30A, S33A, D36A, D37A, Q40A, E47A, or Q32A. The authors discovered that vWF binds the same sub-domain that binds Fc. O'Seaghda et al. define the sub-domain of domain D responsible for binding vWF, but is silent in regard to any use of substitutions in the interacting residues in producing a vaccine antigen.

Gomez et al. (2006) describe the identification of residues responsible for activation of the TNFR1 by using single mutations of F5A, F13A, Y14A, L17A, N21A, I31A, Q32A, and K35A. Gomez et al. is silent in regard to any use of substitutions in the interacting residues in producing a vaccine antigen.

Recombinant affinity tagged Protein A, a polypeptide encompassing the five IgG domains (EDCAB) (Sjodahl, 1977) but lacking the C-terminal Region X (Guss et al., 1984), was purified from recombinant E. coli and used as a vaccine antigen (Stranger-Jones et al., 2006). Because of the attributes of SpA in binding the Fc portion of IgG, a specific humoral immune response to Protein A could not be measured (Stranger-Jones et al., 2006). The inventors have overcome this obstacle through the generation of SpA-DQ9,10K; D36,37A. BALB/c mice immunized with recombinant Protein A (SpA) displayed significant protection against intravenous challenge with S. aureus strains: a 2.951 log reduction in staphylococcal load as compared to the wild-type (P>0.005; Student's t-test) (Stranger-Jones et al., 2006). SpA specific antibodies may cause phagocytic clearance prior to abscess formation and/or impact the formation of the aforementioned eosinophilic barrier in abscesses that separate staphylococcal communities from immune cells since these do not form during infection with Protein A mutant strains. Each of the five SpA domains (i.e., domains formed from three helix bundles designated E, D, A, B, and C) exerts similar binding properties (Jansson et al., 1998). The solution and crystal structure of the domain D has been solved both with and without the Fc and VH3 (Fab) ligands, which bind Protein A in a non-competitive manner at distinct sites (Graille et al., 2000). Mutations in residues known to be involved in IgG binding (FS, Q9, Q10, S11, F13, Y14, L17, N28, 131 and K35) are also required for vWF AI and TNFR1 binding (Cedergren et al., 1993; Gomez et al., 2006; O'Seaghdha et al., 2006), whereas residues important for the VH3 interaction (Q26, G29, F30, S33, D36, D37, Q40, N43, E47) appear to have no impact on the other binding activities (Graille et al., 2000; Jansson et al., 1998). SpA specifically targets a subset of B cells that express VH3 family related IgM on their surface, i.e., VH3 type B cell receptors (Roben et al., 1995). Upon interaction with SpA, these B cells proliferate and commit to apoptosis, leading to preferential and prolonged deletion of innate-like B lymphocytes (i.e., marginal zone B cells and follicular B2 cells) (Goodyear et al., 2003; Goodyear et al., 2004).

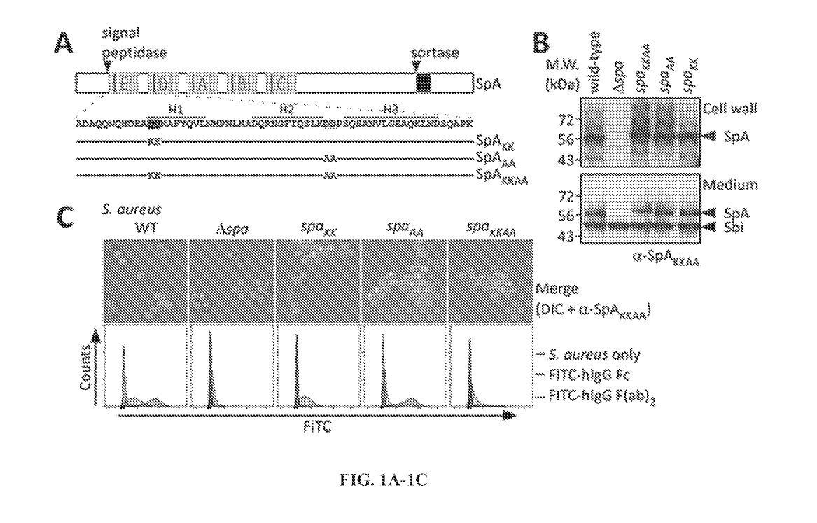

Protein A is synthesized as a precursor in the bacterial cytoplasm and secreted via its YSIRK signal peptide at the cross wall, i.e. the cell division septum of staphylococci (FIG. 1) (DeDent et al., 2007; DeDent et al., 2008). Following cleavage of the C-terminal LPXTG sorting signal, Protein A is anchored to bacterial peptidoglycan crossbridges by sortase A (Mazmanian et al., 1999; Schneewind et al., 1995; Mazmanian et al., 2000). Protein A is the most abundant surface protein of staphylococci; the molecule is expressed by virtually all S. aureus strains (Cespedes et al., 2005; Kennedy et al., 2008; Said-Salim et al., 2003). Staphylococci turn over 15-20% of their cell wall per division cycle (Navarre and Schneewind, 1999). Murine hydrolases cleave the glycan strands and wall peptides of peptidoglycan, thereby releasing Protein A with its attached C-terminal cell wall disaccharide tetrapeptide into the extracellular medium (Ton-That et al., 1999). Thus, by physiological design, Protein A is both anchored to the cell wall and displayed on the bacterial surface but also released into surrounding tissues during host infection (Marraffini et al., 2006).

Protein A captures immunoglobulins on the bacterial surface and this biochemical activity enables staphylococcal escape from host innate and acquired immune responses (Jensen, 1958; Goodyear et al., 2004). Interestingly, region X of Protein A (Guss et al., 1984), a repeat domain that tethers the IgG binding domains to the LPXTG sorting signal/cell wall anchor, is perhaps the most variable portion of the staphylococcal genome (Said-Salim, 2003; Schneewind et al., 1992). Each of the five immunoglobulin binding domains of Protein A (SpA), formed from three helix bundles and designated E, D, A, B, and C, exerts similar structural and functional properties (Sjodahl, 1977; Jansson et al., 1998). The solution and crystal structure of the domain D has been solved both with and without the Fc and V_{H}3 (Fab) ligands, which bind Protein A in a non-competitive manner at distinct sites (Graille 2000).

In the crystal structure complex, the Fab interacts with helix II and helix III of domain D via a surface composed of four VH region β-strands (Graille 2000). The major axis of helix II of domain D is approximately 50° to the orientation of the strands, and the interhelical portion of domain D is most proximal to the CO strand. The site of interaction on Fab is remote from the Ig light chain and the heavy chain constant region. The interaction involves the following domain D residues: Asp-36 of helix II, Asp-37 and Gln-40 in the loop between helix II and helix III and several other residues (Graille 2000). Both interacting surfaces are composed predominantly of polar side chains, with three negatively charged residues on domain D and two positively charged residues on the 2A2 Fab buried by the interaction, providing an overall electrostatic attraction between the two molecules. Of the five polar interactions identified between Fab and domain D, three are between side chains. A salt bridge is formed between Arg-H19 and Asp-36 and two hydrogen bonds are made between Tyr-H59 and Asp-37 and between Asn-H82a and Ser-33. Because of the conservation of Asp-36 and Asp-37 in all five IgG binding domains of Protein A, the inventors mutated these residues.

The SpA-D sites responsible for Fab binding are structurally separate from the domain surface that mediates Fcγ binding. The interaction of Fcγ with domain D primarily involves residues in helix I with lesser involvement of helix II (Gouda et al., 1992; Deisenhofer, 1981). With the exception of the Gln-32, a minor contact in both complexes, none of the residues that mediate the Fcγ interaction are involved in Fab binding. To examine the spatial relationship between these different Ig-binding sites, the SpA domains in these complexes have been superimposed to construct a model of a complex between Fab, the SpA-domain D, and the Fcγ molecule. In this ternary model, Fab and Fcγ form a sandwich about opposite faces of the helix II without evidence of steric hindrance of either interaction. These findings illustrate how, despite its small size (i.e., 56-61 aa), an SpA domain can simultaneously display both activities, explaining experimental evidence that the interactions of Fab with an individual domain are noncompetitive. Residues for the interaction between SpA-D and Fcγ are Gln-9 and Gln-10.

In contrast, occupancy of the Fc portion of IgG on the domain D blocks its interaction with vWF A1 and probably also TNFR1 (O'Seaghdha et al., 2006). Mutations in residues essential for IgG Fc binding (F5, Q9, Q10, S11, F13, Y14, L17, N28, 131 and K35) are also required for vWF A1 and TNFR1 binding (O'Seaghdha et al., 2006; Cedergren et al., 1993; Gomez et al., 2006), whereas residues critical for the VH3 interaction (Q26, G29, F30, S33, D36, D37, Q40, N43, E47) have no impact on the binding activities of IgG Fc, vWF A1 or TNFR1 (Jansson et al., 1998; Graille et al., 2000). The Protein A immunoglobulin Fab binding activity targets a subset of B cells that express V_{H}3 family related IgM on their surface, i.e., these molecules function as VH3type B cell receptors (Roben et al., 1995). Upon interaction with SpA, these B cells rapidly proliferate and then commit to apoptosis, leading to preferential and prolonged deletion of innate-like B lymphocytes (i.e., marginal zone B cells and follicular B2 cells) (Goodyear and Silverman, 2004; Goodyear and Silverman, 2003). More than 40% of circulating B cells are targeted by the Protein A interaction and the V_{H}3 family represents the largest family of human B cell receptors to impart protective humoral responses against pathogens (Goodyear and Silverman, 2004; Goodyear and Silverman, 2003). Thus, Protein A functions analogously to staphylococcal superantigens (Roben et al., 1995), albeit that the latter class of molecules, for example SEB, TSST-1, TSST-2, form complexes with the T cell receptor to inappropriately stimulate host immune responses and thereby precipitating characteristic disease features of staphylococcal infections (Roben et al., 1995; Tiedemann et al., 1995). Together these findings document the contributions of Protein A in establishing staphylococcal infections and in modulating host immune responses.

In sum, Protein A domains can be viewed as displaying two different interfaces for binding with host molecules and any development of Protein A based vaccines must consider the generation of variants that do not perturb host cell signaling, platelet aggregation, sequestration of immunoglobulins or the induction of B cell proliferation and apoptosis. Such Protein A variants should also be useful in analyzing vaccines for the ability of raising antibodies that block the aforementioned SpA activities and occupy the five repeat domains at their dual binding interfaces.

B. Staphylococcal agrA

The agr locus encodes the components of an autoregulatory quorum-sensing system that controls expression of the regulatory RNA molecule RNAIII. Components of this system include agrD, the signaling peptide; agrB, the secretory protein responsible for the export and processing of agrD to its active form; and agrC/agrA, a two-component histidine kinase and response regulator system that detects agrD at critical levels and initiates the expression of those virulence determinants under agr control.

agrA is one member of a family of conserved response regulators with CheY-like receiver domains. These response regulators undergo conformational changes upon the phosphorylation of an aspartate residue by the cognate sensory histidine kinase, allowing them to bind to promoter elements and upregulate transcription. agrA 238 amino acid protein (accession for S. aureus strain Newman is YP_001332980, incorporated herein by reference) of the LytR family of response regulators that recognize a novel element consisting of a pair of direct repeats having a consensus sequence of (TA)([AC)(CA)GTTN(AG)(TG), and separated by a 12- to 13-bp spacer region. Two such elements are found in the P2-P3 intergenic region of RNAIII and the agr operon.

Whereas the agr two-component system has been assumed to follow the canonical quorum-sensing model, the inability to demonstrate binding of agrA to the RNAIII-agr intergenic region led some researchers to question the identification of agrA as a DNA-binding response regulator. However, using purified recombinant agrA in electrophoretic mobility shift assays (EMSAs), agrA has been shown to bind to the P2-P3 region of the agr locus with high affinity. The strongest binding was found to be localized to the pair of direct repeats in the P2 promoter region, with binding to the corresponding pair of repeats in the P3 promoter region being weaker. Phosphorylation of agrA by small phosphodonors had differential effects on binding affinity at the two sites.

C. Staphylococcal srtA

Staphylococcal srtA (surface protein sorting A) is a 206 amino acid polypeptide with an N-terminal hydrophobic domain that functions as a signal peptide/membrane anchor domain. Studies suggest that srtA is assembled in the membrane envelope as a type II membrane protein with its N-terminus in the cytoplasm and the C-terminal end positioned in the cell wall. Strains mutated in srtA are defective in cleaving the sorting signals of protein, fibronectin binding proteins A and B, and clumping factor. As such, srtA is necessary for the cell wall anchoring of certain surface proteins. The accession number for S. aureus Newman srtA is YP_001333460, incorporated herein by reference.

D. Staphylococcal adsA

Adenosine synthase A (adsA), a cell wall-anchored enzyme that converts adenosine monophosphate to adenosine, as a critical virulence factor. Staphylococcal synthesis of adenosine in blood, escape from phagocytic clearance, and subsequent formation of organ abscesses are all dependent on adsA and can be rescued by an exogenous supply of adenosine. adsA homologues exist in anthrax and Bacillus anthracis where it protects from phagocytic clearance. Clearly, staphylococci and other bacterial pathogens exploit the immunomodulatory attributes of adenosine, through adsA, to escape host immune responses.

E. Proteins

The sequences of any of the above proteins may vary from strain to strain and between Staphylococcal species. However, those of skill in the art can identify the corresponding proteins and genes by homology. Also, the term “functionally equivalent codon” is used herein to refer to codons that encode the same amino acid, such as the six codons for arginine or serine, and also refers to codons that encode biologically equivalent amino acids (see Table 1, below). This degeneracy allows variation in nucleic acid sequences when proteins are identical.

TABLE 1
Codon Table

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It also will be understood that proteins and genes may include additional residues, such as additional N- or C-terminal amino acids, or 5′ or 3′ sequences, respectively, natural or synthetic, and yet still be essentially as set forth in one of the proteins disclosed herein, so long as the sequence meets the criteria set forth above, including the maintenance of biological protein activity (e.g., immunogenicity) where protein expression is concerned. The addition of terminal sequences particularly applies to nucleic acid sequences that may, for example, include various non-coding sequences flanking either of the 5′ or 3′ portions of the coding region.

### II. NUCLEIC ACIDS

In certain embodiments, the present invention concerns recombinant polynucleotides encoding for producing, and also encoding, attenuated bacteria of the invention. The nucleic acid sequences for adsA, srtA, agrA and SpA, along with entire genomic sequences are well known to those in the art. The entire sequence for S. aureus Newman is at accession no. NC_009641.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs, including all values and ranges therebetween, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

The nucleic acid segments used in the present invention can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain embodiments, the present invention provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this invention using the methods described herein (e.g., BLAST analysis using standard parameters).

A. Vectors

The term “vector” is used to refer to a carrier nucleic acid molecule into which a heterologous nucleic acid sequence can be inserted. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage. A particular vector in accordance with the present invention is one that carries a transposon.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

1. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain embodiments may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

2. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

3. Selectable and Screenable Markers

In certain embodiments of the invention, cells containing a nucleic acid construct of the present invention may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Of particular interest are markers that create drug sensitivity in the engineered bacteria of the present invention, such as antibiotic markers. While it is viewed that the attenuated strains of the present invention wil be safe for use in subjects, the ability to specifically inhibit these vaccine strains is a useful tool. Various antibiotic resistance markers are well known to those in the art.

B. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (World Wide Web at atcc.org).

C. Mutagenic Procedures

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three. Transposable elements can also be used to “knock in” heterologous sequences.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another. In accordance with the present invention, mutations will be introduced into gram-positive bacteria such as S. aureus using a Himar 1 transposase.

Himar 1 is a “mariner,” one of a widespread and diverse family of animal transposons. Himar 1 is derived from Haematobia irritans. This transposase can reproduce transposition faithfully in an in vitro inter-plasmid transposition reaction. It binds to the inverted terminal repeat sequences of its cognate transposon and mediates 5′ and 3′ cleavage of the element termini. It functions independent of species-specific host factors, which explains the broad distribution of mariners and why they are capable of horizontal transfer between species (Lampe et al., 1996).

U.S. Patent Application Publication No. 2006/0275905 also discloses suitable mutagenic procedures and is hereby incorporated by reference.

### IV. IMMUNE RESPONSE AND ASSAYS

As discussed above, the invention concerns evoking or inducing an immune response in a subject. In one embodiment, the immune response can protect against or treat a subject having, suspected of having, or at risk of developing an infection or related disease, particularly those related to staphylococci. One use of the immunogenic compositions of the invention is to prevent nosocomial infections by inoculating a subject prior to undergoing procedures in a hospital or other environment having an increased risk of infection.

Staphylococcal infections progress through several different stages. For example, the staphylococcal life cycle involves commensal colonization, initiation of infection by accessing adjoining tissues or the bloodstream, and/or anaerobic multiplication in the blood. The interplay between S. aureus virulence determinants and the host defense mechanisms can induce complications such as endocarditis, metastatic abscess formation, and sepsis syndrome. Different molecules on the surface of the bacterium are involved in different steps of the infection cycle. Combinations of certain antigens can elicit an immune response which protects against multiple stages of staphylococcal infection. The effectiveness of the immune response can be measured either in animal model assays and/or using an opsonophagocytic assay.

A. Immunoassays

The present invention includes the implementation of serological assays to evaluate whether and to what extent an immune response is induced or evoked by compositions of the invention. There are many types of immunoassays that can be implemented. Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain preferred immunoassays are the various types of enzyme linked immunosorbent assays (ELISAs) and radioimmunoassays (RIA) known in the art. Immunohistochemical detection using tissue sections is also particularly useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

B. Diagnosis of Bacterial Infection

In addition to the use of proteins, polypeptides, and/or peptides, as well as antibodies binding these polypeptides, proteins, and/or peptides, to treat or prevent infection as described above, the present invention contemplates the use of these polypeptides, proteins, peptides, and/or antibodies in a variety of ways, including the detection of the presence of staphylococcus to diagnose an infection, whether in a patient or on medical equipment which may also become infected. In accordance with the invention, a preferred method of detecting the presence of infections involves the steps of obtaining a sample suspected of being infected by one or more staphylococcal bacteria species or strains, such as a sample taken from an individual, for example, from one's blood, saliva, tissues, bone, muscle, cartilage, or skin. Following isolation of the sample, diagnostic assays utilizing the polypeptides, proteins, peptides, and/or antibodies of the present invention may be carried out to detect the presence of staphylococci, and such assay techniques for determining such presence in a sample are well known to those skilled in the art and include methods such as radioimmunoassay, western blot analysis and ELISA assays. In general, in accordance with the invention, a method of diagnosing an infection is contemplated wherein a sample suspected of being infected with staphylococci has added to it the polypeptide, protein, peptide, antibody, or monoclonal antibody in accordance with the present invention, and staphylococci are indicated by antibody binding to the polypeptides, proteins, and/or peptides, or polypeptides, proteins, and/or peptides binding to the antibodies in the sample.

Accordingly, antibodies produced in accordance with the invention may be used for the prevention of infection from staphylococcal bacteria (i.e., passive immunization), for the treatment of an ongoing infection, or for use as research tools. The term “antibodies” as used herein includes monoclonal, polyclonal, chimeric, single chain, bispecific, simianized, and humanized or primatized antibodies as well as Fab fragments, such as those fragments which maintain the binding specificity of the antibodies, including the products of an Fab immunoglobulin expression library. Accordingly, the invention contemplates the use of single chains such as the variable heavy and light chains of the antibodies. Generation of any of these types of antibodies or antibody fragments is well known to those skilled in the art. Specific examples of the generation of an antibody to a bacterial protein can be found in U.S. Patent Application Pub. No. 20030153022, which is incorporated herein by reference in its entirety.

C. Protective Immunity

In some embodiments of the invention, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, carbohydrate, or polypeptide of the invention in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens present in the same, related, or different microbes or organisms, such as gram-positive bacteria, gram-negative bacteria, including but not limited to staphylococcus bacteria.

Passive immunity may be imparted to a patient or subject by administering to the patient immunoglobulins (Ig) and/or other immune factors obtained from a donor or other non-patient source having a known immunoreactivity. In other aspects, an antigenic composition of the present invention can be administered to a subject who then acts as a source or donor for globulin, produced in response to challenge with the antigenic composition (“hyperimmune globulin”), that contains antibodies directed against Staphylococcus or other organism. A subject thus treated would donate plasma from which hyperimmune globulin would then be obtained, via conventional plasma-fractionation methodology, and administered to another subject in order to impart resistance against or to treat staphylococcus infection. Hyperimmune globulins according to the invention are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996). Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by 3H-thymidine incorporation by primed T cells in response to an epitope (Burke et al., 1994), by antigen-dependent killing (cytotoxic T lymphocyte assay, Tigges et al., 1996) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

As used herein and in the claims, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

As used herein the terms “immunogenic agent” or “immunogen” or “antigen” are used interchangeably to describe a molecule capable of inducing an immunological response against itself on administration to a recipient, either alone, in conjunction with an adjuvant, or presented on a display vehicle.

D. Treatment Methods

A method of the present invention includes treatment for a disease or condition caused by a staphylococcus pathogen. A bacterium or vaccine of the present invention can be administered to induce an immune response in a person infected with staphylococcus, suspected of having been exposed to staphylococcus, or at risk of such exposure. Methods may be employed with respect to individuals who have tested positive for exposure to staphylococcus or who are deemed to be at risk for infection based on possible exposure.

In particular, the invention encompasses a method of treatment for staphylococcal infection, particularly hospital acquired nosocomial infections. The bacteria and vaccines of the invention are particularly advantageous to use in cases of elective surgery. Such patients will know the date of surgery in advance and could be inoculated in advance. The bacteria and vaccines of the invention are also advantageous to use to inoculate health care workers.

In some embodiments, the treatment is administered in the presence of biological response modifiers. Furthermore, in some examples, treatment comprises administration of other agents commonly used against bacterial infection, such as one or more antibiotics.

The use of vaccines, discussed below, to treat or prevent infections (active immunization) is specifically contemplated, as is the transfer of immune effectors from a vaccinated patient to another subject (passive immunization).

E. Combination Therapy

The compositions and related methods of the present invention, particularly administration of a bacterium or vaccine, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a vaccine and/or therapy is used in conjunction with antibacterial treatment. Alternatively, the vaccine therapy may precede or follow the other agent treatment by intervals ranging from minutes to weeks. In embodiments where the other agents and/or vaccine are administered separately, one would generally ensure that a significant period of time did not expire between the time of each delivery, such that the agent and vaccine composition would still be able to exert an advantageously combined effect on the subject. In such instances, it is contemplated that one may administer both modalities within about 12-24 h of each other or within about 6-12 h of each other. In some situations, it may be desirable to extend the time period for administration significantly, where several days (2, 3, 4, 5, 6 or 7) to several weeks (1, 2, 3, 4, 5, 6, 7 or 8) lapse between the respective administrations.

Various combinations may be employed, for example, where the vaccine therapy is “A” and the other therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of the immunogenic compositions of the present invention to a patient/subject will follow general protocols for the administration of such compounds, taking into account the toxicity, if any, of the vaccine or other compositions described herein. It is expected that the treatment cycles would be repeated as necessary. It also is contemplated that various standard therapies, such as hydration, may be applied in combination with the described therapy. Secondary agents include antibiotics and polyclonal antisera (WO00/15238, WO00/12132) or monoclonal antibodies against lipoteichoic acid (WO98/57994).

### V. VACCINES AND OTHER PHARMACEUTICAL COMPOSITIONS AND ADMINISTRATION

A. Vaccines

The present invention includes methods for preventing or ameliorating staphylococcal infections, particularly hospital acquired nosocomial infections. As such, the invention contemplates vaccines for use in both active and passive immunization embodiments. The bacteria and vaccines are described elsewhere in this document.

The preparation of vaccines is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkylene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms of active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application within a solid physiologically acceptable base or in a physiologically acceptable dispersion, parenterally, by injection and the like. The dosage of the vaccine will depend on the route of administration and will vary according to the size and health of the subject.

In certain instances, it will be desirable to have multiple administrations of the vaccine, e.g., 2, 3, 4, 5, 6 or more administrations. The vaccinations can be at 1, 2, 3, 4, 5, 6, 7, 8, to 5, 6, 7, 8, 9, 10, 11, 12 twelve week intervals, including all ranges there between. Periodic boosters at intervals of 1-5 years will be desirable to maintain protective levels of the antibodies. The course of the immunization may be followed by assays for antibodies against the antigens, as described in U.S. Pat. Nos. 3,791,932; 4,174,384 and 3,949,064.

The immunogenicity of polypeptide or peptide compositions can be enhanced by the use of non-specific stimulators of the immune response, known as biological response modifiers. Such agents include all acceptable immunostimulatory compounds, such as cytokines, toxins, or synthetic compositions, including adjuvants that can (1) trap the antigen in the body to cause a slow release; (2) attract cells involved in the immune response to the site of administration; (3) induce proliferation or activation of immune system cells; or (4) improve the spread of the antigen throughout the subject's body.

Biological response modifiers include, but are not limited to, oil-in-water emulsions, water-in-oil emulsions, mineral salts, polynucleotides, and natural substances, and specific examples that may be used include IL-1, IL-2, IL-4, IL-7, IL-12, -interferon, GMCSP, BCG, aluminum salts, such as aluminum hydroxide or other aluminum compound, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM), and cell wall skeleton (CWS) in a 2% squalene/Tween 80 emulsion. MHC antigens may even be used. Others agents or methods are exemplified in U.S. Pat. Nos. 6,814,971, 5,084,269, 6,656,462, each of which is incorporated herein by reference).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Examples of and often preferred adjuvants include complete Freund's adjuvant (a non-specific stimulator of the immune response containing killed Mycobacterium tuberculosis), incomplete Freund's adjuvants, and aluminum hydroxide.

In some aspects, it is preferred that the agent be selected to be a preferential inducer of either a Th1 or a Th2 type of response. High levels of Th1-type cytokines tend to favor the induction of cell mediated immune responses to a given antigen, while high levels of Th2-type cytokines tend to favor the induction of humoral immune responses to the antigen.

The distinction of Th1 and Th2-type immune response is not absolute. In reality an individual will support an immune response which is described as being predominantly Th1 or predominantly Th2. However, it is often convenient to consider the families of cytokines in terms of that described in murine CD4+ T cell clones by Mosmann and Coffman (Mosmann, and Coffman, 1989). Traditionally, Th1-type responses are associated with the production of the INF-γ and IL-2 cytokines by T-lymphocytes. Other cytokines often directly associated with the induction of Th1-type immune responses are not produced by T-cells, such as IL-12. In contrast, Th2-type responses are associated with the secretion of IL-4, IL-5, IL-6, IL-10.

Other than traditional adjuvants, biologic response modifiers (BRM) include agents shown to upregulate T cell immunity or downregulate suppresser cell activity. Such BRMs include, but are not limited to, Cimetidine (CIM; 1200 mg/d) (Smith/Kline, PA); or low-dose Cyclophosphamide (CYP; 300 mg/m²) (Johnson/Mead, NJ) and cytokines such as γ-interferon, IL-2, or IL-12 or genes encoding proteins involved in immune helper functions, such as B-7.

B. General Pharmaceutical Compositions

In some embodiments, pharmaceutical compositions are administered to a subject. Different aspects of the present invention involve administering an effective amount of a composition to a subject. Additionally, such compounds can be administered in combination with an antibiotic or an antibacterial. Such compositions will generally be dissolved or dispersed in a pharmaceutically acceptable carrier or aqueous medium.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds of the present invention can be formulated for parenteral administration, e.g., formulated for injection via the intravenous, intramuscular, sub-cutaneous, or even intraperitoneal routes. The preparation of an aqueous composition that contains a compound or compounds that increase the expression of an MHC class I molecule will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions according to the present invention will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

For parenteral administration in an aqueous solution, for example, the solution should be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

C. Antibodies and Passive Immunization

Another aspect of the invention is a method of preparing an immunoglobulin or serum for use in prevention or treatment of staphylococcal infection comprising the steps of immunizing a recipient or donor with the vaccine of the invention and isolating immunoglobulin from the recipient or donor. An immunoglobulin or serum prepared by this method is a further aspect of the invention. A pharmaceutical composition comprising the immunoglobulin of the invention and a pharmaceutically acceptable carrier is a further aspect of the invention which could be used in the manufacture of a medicament for the treatment or prevention of staphylococcal disease. A method for treatment or prevention of staphylococcal infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies.

The antibodies can be isolated to the extent desired by well known techniques such as affinity chromatography (Harlow and Lane, 1988). Antibodies can include antiserum preparations from a variety of commonly used animals, e.g. goats, primates, donkeys, swine, horses, guinea pigs, rats or man.

An immunoglobulin produced in accordance with the present invention can include whole antibodies, antibody fragments or subfragments. Antibodies can be whole immunoglobulins of any class (e.g., IgG, IgM, IgA, IgD or IgE), chimeric antibodies or hybrid antibodies with dual specificity to two or more antigens of the invention. They may also be fragments (e.g., F(ab′)2, Fab′, Fab, Fv and the like) including hybrid fragments. An immunoglobulin also includes natural, synthetic, or genetically engineered proteins that act like an antibody by binding to specific antigens to form a complex.

A vaccine of the present invention can be administered to a recipient who then acts as a source of immunoglobulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat staphylococcal infection. Hyperimmune globulins of the invention are particularly useful for treatment or prevention of staphylococcal disease in infants, immune compromised individuals, or where treatment is required and there is no time for the individual to produce antibodies in response to vaccination.

An additional aspect of the invention is a pharmaceutical composition comprising two of more monoclonal antibodies (or fragments thereof; preferably human or humanised) reactive against at least two constituents of the immunogenic composition of the invention, which could be used to treat or prevent infection by Gram positive bacteria, preferably staphylococci, more preferably S. aureus or S. epidermidis. Such pharmaceutical compositions comprise monoclonal antibodies that can be whole immunoglobulins of any class, chimeric antibodies, or hybrid antibodies with specificity to two or more antigens of the invention. They may also be fragments (e.g., F(ab′)2, Fab′, Fab, Fv and the like) including hybrid fragments.

Methods of making monoclonal antibodies are well known in the art and can include the fusion of splenocytes with myeloma cells (Kohler and Milstein, 1975; Harlow and Lane, 1988). Alternatively, monoclonal Fv fragments can be obtained by screening a suitable phage display library (Vaughan et al., 1998). Monoclonal antibodies may be humanized or part humanized by known methods.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Bacterial strains. S. aureus strains Newman and its variants or USA300 LAC were grown in tryptic soy broth (TSB) or agar at 37° C. Escherichia coli strains DH5a and BL21(DE3) were grown in Luria broth (LB) or agar at 37° C. Ampicillin (100 μg·mL⁻¹ for E. coli), spectinomycin (200 μg·mL⁻¹ for S. aureus) or erythromycin (20 μg·mL⁻¹ for S. aureus) were used for plasmid (pET15b+), mutant allele selection (Δspa) or transposon selection (Δsbi).

S. aureus spa mutants. Two 1 kb DNA sequence segments upstream and downstream of the spa gene were amplified from chromosome of S. aureus Newman (Baba, 2007) with primers:

ext1F
(SEQ ID NO. 7)
(5′GGGGACCACTTTGTACAAGAAAGCTGGGTCATTTAAGAAGATTG
TTTCAGATTTATG-3′),
ext1R
(SEQ ID NO. 8)
(5′-ATTTGTAAAGTCATCATAATATAACGAATTATGTATTGCAATA
CTAAAATC-3′),
and
ext2F
(SEQ ID NO. 9)
(5′-CGTCGCGAACTATAATAAAAACAAACAATACACAACGATAGAT
ATC-3′),
ext2R
(SEQ ID NO. 10)
(5′GGGGACAAGTTTGTACAAAAAAGCAGGCAACGAACGCCTAAAGA
AATTGTCTTTGC-3′).

The DNA sequences of spa_{KKAA}, spa_{AA} and spa_{KK} mutants were previously described (Kim, 2012). These sequences were amplified using the primers spaF (CATAATTCGTTATATTATGATGACTTTACAAATACATACAGGG) (SEQ ID NO. 11) and spaR (GTATTGTTTGTTTTTATTATAGTTCGCGACGACGTCCA) (SEQ ID NO. 12). For each construct, mutant spa genes and their two flanking region were fused together in a subsequent PCR reaction. The final PCR products were cloned onto pKOR1 (Bae, 2005) using the BP clonase II kit (Invitrogen). Plasmids were electroporated into the S. aureus Δspa variant and temperature shifted to 42° C., blocking replication of plasmids and promoting their insertion into the chromosome. Growth at 30° C. was used to promote allelic replacement. Mutations in the spa genes were verified by DNA sequencing of PCR amplification products.

Purification of Protein A.

E. coli BL21 (DE3) harboring pET15b+ plasmids for the expression of His-tagged wild-type SpA, SpA_{KK}, SpA_{AA} and SpA_{KKAA}, (Kim, 2012) were grown overnight, diluted 1:100 into fresh media and grown at 37° C. to A₆₀₀ 0.5. Cultures were induced with 1 mM isopropyl β-D-1-thiogalatopyranoside (IPTG) and grown for an additional three hours. Bacterial cells were sedimented by centrifugation, suspended in column buffer (50 mM Tris-HCl (pH 7.5), 150 mM NaCl) and disrupted with a French pressure cell at 14,000 psi. Lysates were cleared of membrane and insoluble components by ultracentrifugation at 40,000×g. Cleared lysates were subjected to nickel-nitrilotriacetic acid (Ni-NTA) affinity chromatography, and proteins were eluted in column buffer containing successively higher concentrations of imidazole (100-500 mM). Eluates were dialyzed with PBS, treated with Triton-X114 to remove endotoxin and again dialyzed with PBS. Protein concentrations were determined by bicinchoninic acid (BCA) assay (Thermo Scientific). Purity was verified by Coomassie-stained SDS-PAGE.

Affinity Chromatography of Immunoglobulin.

Purified His₆-tagged SpA, SpA_{AA}, SpA_{KK} and SpA_{KKAA} were immobilized on nickel-nitrilotriacetic acid (Ni-NTA) sepharose, washed and incubated with human IgG, Fc, F(ab)₂ fragments in 50 mM Tris-HCl (pH 7.5), 150 mM NaCl buffer. After washing, proteins were eluted with 500 mM imidazole and analyzed by SDS-PAGE.

Enzyme Linked Immuno-Sorbent Assay.

To determine antigen specific serum IgG, recombinant purified staphylococcal antigens (SpA_{KKAA}, ClfA, FnBPB, IsdB, Coa, and Hla) (Kim, 2010) were used to coat ELISA plates (NUNC Maxisorp) at 1 μg·mL⁻¹ in 0.1 M carbonate buffer (pH 9.5 at 4° C. overnight). The following day, plates were blocked and incubated with serially diluted sera. Plates were incubated with HRP-conjugated secondary antibody specific to mouse IgG (or isotype specific antibodies) and developed using OptEIA reagent.

Protein a Expression in S. aureus.

Overnight cultures of staphylococci were diluted 1:100 and grown at 37° C. with shaking to A₆₀₀ 2. Fractionation of staphylococci into medium and cell wall compartments followed a previously established procedure (Mazmanian, 2000). Briefly, bacteria were centrifuged and the extracellular medium in supernatant was precipitated with 5% TCA. The pellet was suspended in TSM [50 mM Tris (pH 7.5), 500 mM sucrose, and 10 mM MgCl₂ with 100 μg·mL⁻¹ lysostaphin] and incubated at 37° C. to solubilize the cell wall envelope. The resulting protoplasts were sedimented by centrifugation, and the supernatant was precipitated with TCA (cell wall fraction). TCA precipitated proteins were washed in acetone, dried, solubilized in sample buffer and separated on SDS-PAGE. Proteins were electro-transferred to PDVF membrane and analyzed by immunoblotting using affinity-purified rabbit α-SpA_{KKAA} antibody (Kim, 2010).

Circular Dichroism Spectroscopy.

Far ultraviolet (UV) CD spectra of purified SpA, SpA_{AA}, SpA_{KK} and SpA_{KKAA} in 10 mM phosphate buffer (pH 7.2), 50 mM Na₂SO₄ were recorded on a AVIV 202 CD Spectrometer (University of Chicago Biophysics Core Facility) at room temperature.

Immunofluorescence Microscopy.

Overnight cultures of staphylococci were diluted 1:100 and grown at 37° C. with shaking to A₆₀₀ 0.7. Bacteria were centrifuged, washed, fixed with glutaraldehyde and blocked. Cells were incubated with affinity purified α-SpA_{KKAA} rabbit IgG for 1 hour, washed, incubated with Alexafluor 647 conjugated goat α-rabbit IgG (Invitrogen) and washed in PBS. Bacteria were settled in poly-lysine treated glass coverslips and then applied to glass coverslips containing a drop of SlowFade anti-fading reagent (Invitrogen). Images were captured on a Leica SP5 Tanden Scanner Spectral 2-Photon confocal microscope at the University of Chicago Light Microscopy Core Facility.

Flow Cytometry.

Overnight cultures of staphylococci grown in TSB were diluted 1:100 and grown at 37° C. with shaking to A₆₀₀ 0.6. Bacteria were centrifuged, washed, fixed and blocked. To analyze immunoglobulin binding to staphylococci, cells were incubated with FITC-conjugated Fcγ or F(ab)₂ fragments of human IgG (1:250), washed in 1% BSA/PBS. To examine the presence of natural antibodies against S. aureus in naïve mouse serum, staphylococci were incubated with dilutions of naïve mouse sera (C57BL/6 and BALB/c, Taconic) for 30 minutes at room temperature with slow rotation. Cells were washed, incubated with PE conjugated goat α-mouse IgM or FITC conjugated goat α-mouse IgG (1:250) and washed in 1% BSA/PBS.

Active Immunization.

3 week old, female BALB/c mice (Charles River Laboratories) were immunized with 50 μg of SpA or its variants emulsified in complete Freund's adjuvant (CFA, Difco) and boosted with 50 μg of the same antigen emulsified in incomplete Freund's adjuvant (IFA) 11 days following the first immunization. On day 21, mice were bled and serum recovered for ELISA experiments.

Mouse Renal Abscess Model.

Overnight cultures of S. aureus Newman (wild-type) and its Δspa, spa_{AA}, spa_{KK} and spa_{KKAA} variants were diluted 1:100 into fresh TSB and grown for 2 hours at 37° C. Staphylococci were sedimented, washed and suspended in PBS to the desired bacterial concentration. Inocula were quantified by spreading sample aliquots on TSA and enumerating CFU. BALB/c mice were anesthetized via intraperitoneal injection with 100 mg·ml⁻¹ ketamine and 20 mg·ml⁻¹ xylazine per kilogram of body weight. Mice were infected by injection with 1×10⁷ CFU of S. aureus Newman or its variants into the periorbital venous sinus of the right eye. On day 15 or 28 following infection, mice were euthanized by CO₂ inhalation and cervical dislocation. Both kidneys were removed, and the staphylococcal load in one organ was analyzed by homogenizing renal tissue with PBS, 0.1% Triton X-100. Serial dilutions of homogenate were spread on TSA and incubated for colony formation. The remaining organ was examined by histopathology. Briefly, kidneys were fixed in 10% formalin for 24 hours at room temperature. Tissues were embedded in paraffin, thin-sectioned, stained with hematoxylin-eosin, and inspected by light microscopy to enumerate abscess lesions. Immune serum samples collected at 15 days post infection were examined by ELISA against the staphylococcal antigen matrix. To examine whether attenuated strains elicit protective efficacy, animals were infected with spa_{KKAA} for 15 days and treated with daptomycin at 10 mg·kg⁻¹ for 4 days. Three days after the last injection of daptomycin, animals were challenged with 5×10⁷ CFU of S. aureus USA300 and monitored for 10 days. All mouse experiments were performed at least twice and conducted in accordance with the institutional guidelines following experimental protocol review and approval by the Institutional Biosafety Committee (IBC) and the Institutional Animal Care and Use Committee (IACUC) at the University of Chicago.

Staphylococcal Survival in Blood In Vivo.

Overnight cultures of S. aureus Newman and its Δspa, spa_{AA}, spa_{KK} or spa_{KKAA} variants were diluted 1:100 into fresh media and grown for 2 hours at 37° C. Staphylococci were sedimented by centrifugation, washed and suspended in PBS to the desired bacterial concentration. Inocula were quantified by spreading sample aliquots on TSA and enumerating the colonies that formed upon incubation. C57BL/6J and B6.129S2-Ighm^{tmlCgn}/J (μMT) mice (Jackson Laboratory) were anesthetized via intraperitoneal injection with 100 mg·ml⁻¹ ketamine and 20 mg·ml⁻¹ xylazine per kilogram of body weight. Mice were infected by injection with 1×10⁶ CFU of S. aureus into the periorbital venous sinus of the right eye. At 30 minutes post infection, mice were euthanized by CO₂ inhalation. Blood was collected by cardiac puncture, and mixed with 2% saponin/PBS in 1:1. Dilutions of staphylococci were plated on agar for colony formation.

Staphylococcal Survival in Blood In Vitro.

Whole blood was collected from mice by cardiac puncture and coagulation inhibited with 10 μg·ml⁻¹ lepirudin. 50 μl of 5×10⁶ CFU·ml⁻¹ of S. aureus Newman or variants were mixed with 950 μl of mouse blood. Samples were incubated at 37° C. with slow rotation for 30 minutes and then incubated on ice with 1% saponin/PBS to lyse eukaryotic cells. Dilutions of staphylococci were plated on agar for colony formation.

Statistical Analysis.

Bacterial loads and number of abscesses in experimental animal infection model were analyzed with the two-tailed Mann-Whitney test to measure statistical significance. Unpaired two-tailed Student's t-tests were performed to analyze the statistical significance of ELISA data and blood survival data. All data were analyzed by Prism (GraphPad Software, Inc.) and P values less than 0.05 were deemed significant.

### Example 2

### Results

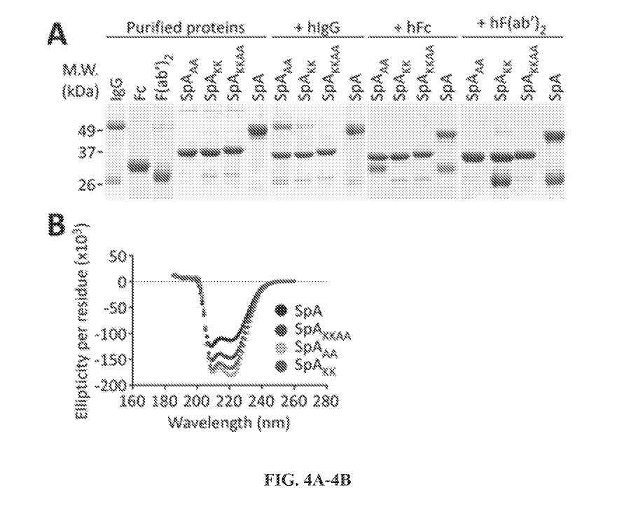

Guided by the structural analysis of protein A co-crystallized with Fcγ or Fab (Deisenhofer, 1978; Graille, 2000), the inventors generated S. aureus strains and recombinant SpA variants with amino acid substitutions at residues 9-10 (Gln⁹Lys, Gln¹⁰Lys) and/or 36-37 (Asp³⁶Ala, Asp³⁷Ala) of all five IgBDs (FIG. 1A). These substitutions abolished binding of recombinant SpA to Fcγ (SpA_{KK}), Fab (SpA_{AA}) or Fcγ and Fab (SpA_{KKAA})(FIG. 4). When expressed in S. aureus and probed by immunoblotting with specific antibodies, similar amounts wild-type and mutant SpA were detected in the bacterial envelope and in the extracellular medium of S. aureus cultures (FIG. 1BC). The secretion of Sbi, a second staphylococcal IgG binding protein with homology to SpA (Zhang, 1998), was not impacted by spa mutations (FIG. 1B). Wild-type S. aureus binds to both the Fcγ and F(ab)₂ domains of human immunoglobulin (FIG. 1C). Fcγ binding was abolished in the spa_{KK} and spa_{KKAA} variants, but not in the spa_{AA} mutant (FIG. 1C). The binding of human F(ab)₂ fragments to spa_{AA} and spa_{AA} mutants was reduced, but not affected in the spa_{KK} variant (FIG. 1C). The residual amount of F(ab)₂ fragment binding to the spa_{KKAA} mutant is based on antibody recognition of staphylococcal surface antigens, as similar binding activities were observed for S. aureus mutants lacking the entire spa gene (Δspa) (FIG. 1C).

The virulence of wild-type and spa mutant staphylococci was assessed by intravenous injection of 1×10⁷ colony forming units (CFU) into naïve BALB/c mice. Animals were euthanized 15 days after challenge, necropsied and staphylococcal load and abscess formation in renal tissues determined (Table 1). The spa_{KKAA} variant was attenuated for both abscess formation in renal tissues and staphylococcal load, similar to the Δspa mutant (Table 1). The spa_{AA} and spa_{KK} mutants displayed an intermediate phenotype for the staphylococcal load. Further, the spa_{KK} mutant was defective for abscess formation, whereas the spa_{AA} variant was not (Table 1). These data indicate that both biological activities of protein A, Ig Fcγ binding and Fab crosslinking, contribute to the pathogenesis of S. aureus infections in mice. Moreover, protein A-dependent B cell superantigen activity is not required for the formation of staphylococcal abscess lesions in naïve mice.

TABLE 1
Virulence defects of S. aureus variants expressing mutant protein A.
BALB/c mice were infected with 1 × 10⁷ CFU wild-type,
Δspa, spa_{KK}, spa_{AA} or spa_{KKAA} mutant S. aureus
Newman. At 15 days post infection, animals were euthanized,
necropsied and bacterial load (log₁₀CFU g⁻¹)
and number of abscess lesions in kidney tissues determined.

| | | Staphylococcal load | | | Abscess formation | |
|---|---|---|---|---|---|---|
| S. | | ^{b}log₁₀CFU | | ^{d}Re- | ^{e}Number of | |
| aureus | ^{a}n | g⁻¹ | ^{c}P value | duction | abscesses | ^{c}P value |
| wild- | 18 | 6.20 ± 0.43 | — | — | 8.50 ± 1.75 | — |
| type | | | | | | |
| Δspa | 20 | 4.49 ± 0.41 | 0.0017 | 1.71 | 2.25 ± 0.71 | 0.0346 |
| spa_{KK} | 20 | 5.29 ± 0.41 | 0.0924 | 0.91 | 2.50 ± 0.74 | 0.0315 |
| spa_{AA} | 19 | 4.70 ± 0.53 | 0.0528 | 1.50 | 5.11 ± 1.41 | 0.2502 |
| spa_{KKAA} | 20 | 4.24 ± 0.47 | 0.0069 | 1.96 | 2.85 ± 0.98 | 0.0206 |

^{a}Number of 6 week old, female BALB/c mice per study.
^{b}Means (±SEM) of staphylococcal load calculated as log₁₀ CFU g⁻¹ in homogenized renal tissues 15 days following infection; limit of detection: 1.99 log₁₀CFU g⁻¹.
^{c}Statistical significance was calculated with the unpaired two-tailed Mann-Whitney test and P-values recorded.
^{d}Reduction in bacterial load calculated as log₁₀ CFU g⁻¹.
^{e}Histopathology of hematoxylin-eosin stained, thin sectioned kidneys revealed the mean number of abscesses per kidney (±SEM).

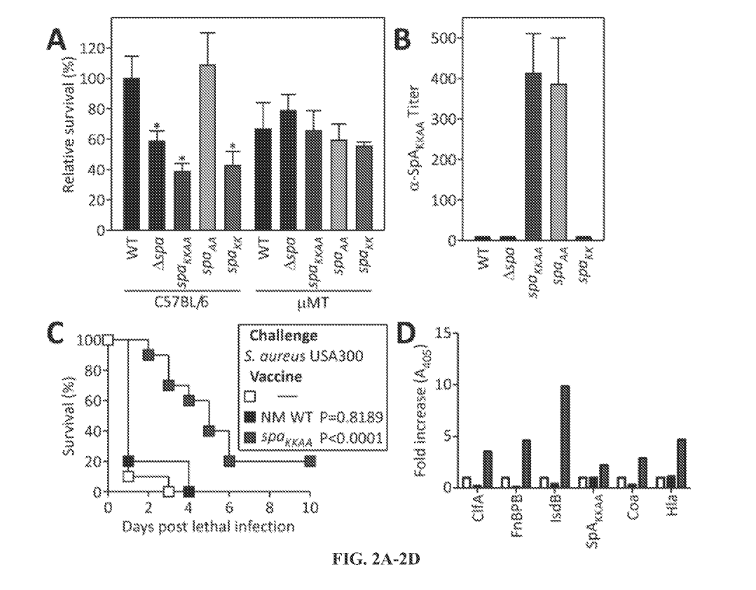

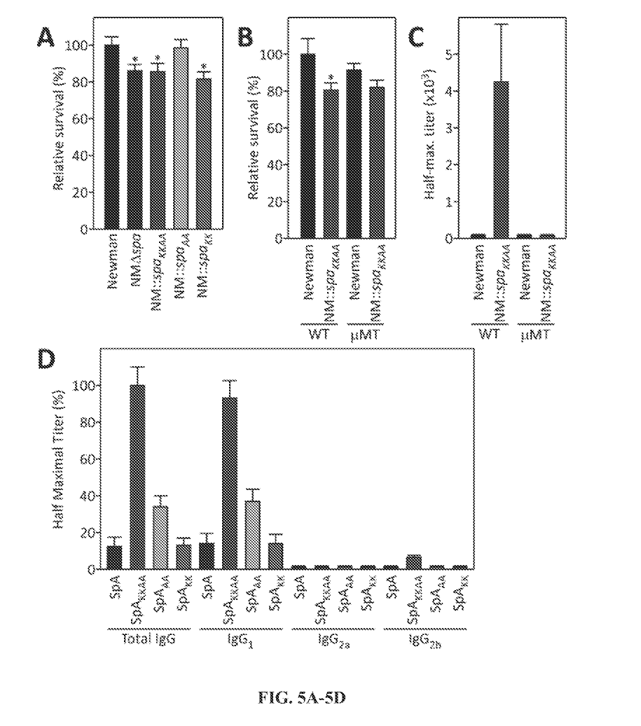

To further explore the contributions of protein A to S. aureus disease, the inventors infected mice by intravenous inoculation into the retroorbital plexus, removed blood samples after 30 min by cardiac puncture and enumerated staphylococcal CFU. Wild-type and spa_{AA} mutant S. aureus survived in the bloodstream of naïve mice, whereas the Δspa, spa_{KK} and spa_{KKAA} variants were killed (FIG. 2A). Compared to wild-type C57BL/6 mice, the survival of wild-type S. aureus was reduced in the blood stream of μMT mice, which lack both mature B cells and immunoglobulin (FIG. 2A). Further, no significant difference in blood stream survival in μMT mice was detected between wild-type and spa_{KKAA} mutant S. aureus (FIG. 2A). Mice infected with the spa_{AA} and spa_{KKAA} mutants (but not animals infected with wild-type, Δspa or spa_{KK} variants) developed IgG antibodies against protein A (SpA_{KKAA}) (FIG. 2B). Compared to naïve mice or animals with a history of wild-type S. aureus infection, mice that had been infected with the spa_{KKAA} variant and treated with daptomycin acquired protection from lethal challenge with S. aureus LAC, the current epidemic MRSA (USA300) strain in the United States (Kennedy, 2008) (FIG. 2C). Mice infected with the spa_{KKAA} variant developed IgG antibodies against six secreted virulence factors that represent leading vaccine candidates: ClfA, FnBPB, IsdB, Coa, Hla and SpA (FIG. 2D) (Rivas, 2008; Cheng, 2010). These results suggest that prior infection of mice with the spa_{KKAA} variant, which does not cause disease (Table 2), elicits antibodies against S. aureus protective antigens and raises protective immunity in mice against highly virulent MRSA strains. The development of protective immunity by the spa_{KKAA} variant is due to the loss of protein A-dependent B cell superantigen activity. In support of this hypothesis, immunization of mice with purified SpA_{KKAA} elicited high titer specific antibodies and IgG class switching (IgG1 and IgG2b) (FIG. 5). This was not observed when immunizing mice with either wild-type SpA or SpA_{KK}. SpA_{AA} immunization elicited specific antibodies, however IgG titers were lower and IgG class switching did not occur (FIG. 5).

TABLE 2
S. aureus spa_{KKAA} as a live-attenuated whole cell vaccine against
MRSA

| | Staphylococcal load in renal tissue | | |
|---|---|---|---|
| ^{a}Vaccine | ^{b}log₁₀CFU g⁻¹ | ^{c}Body weight | ^{d}P value |
| Naïve | — | 18.09 ± 0.20 | — |
| spa_{KKAA} | 2.91 ± 0.54 | 18.38 ± 0.46 | 0.4584 |

^{a}BALB/c mice (6 weeks old female, n = 10) were infected by intravenous inoculation with 1 × 10⁷ CFU of S. aureus spa_{KKAA} or left uninfected (naïve). At 15 day post infection, animals were treated with intraperitoneal injections of daptomycin at 10 mg · kg⁻¹ for four days. On day 22, mice were weighed, euthanized, necropsied and staphylococcal load and abscess formation were measured in kidneys of infected animals.
^{b}Means (±SEM) of staphylococcal load calculated as log₁₀ CFU g⁻¹ in homogenized renal tissues 22 days following infection with limit of detection at 1.99 log₁₀CFU g⁻¹.
^{c}Means (±SEM) of body weights measured at day 22.
^{d}Statistical significance of body weight measurements was calculated with the unpaired two-tailed student's t-test and P-values recorded.

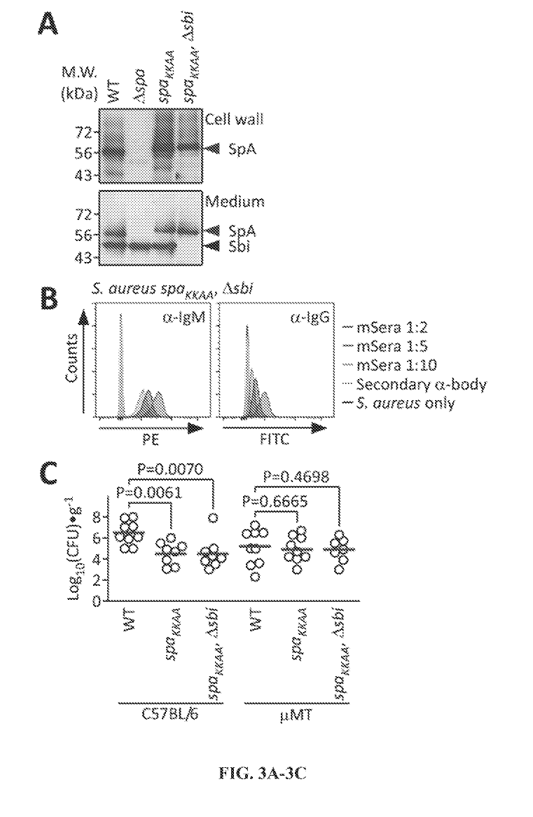

The inventors asked whether Sbi, which binds IgG Fcγ (Zhang, 1998) as well as complement factors H and C3b (Haupt, 2008), contributes to S. aureus escape from host immune surveillance by generating the spa_{KKAA}/sbi mutant (FIG. 3A). When subjected to flow cytometry with mouse IgM and IgG, antibodies of both Ig types bound to the surface of the spa_{KKAA}/sbi mutant (FIG. 3B). These natural antibodies against S. aureus were detected in sera from naïve BALB/c and C57BL/6 animals, but not in μMT mice. To discern whether natural antibodies provide protection against S. aureus, C57BL/6 and μMT mice were infected with wild-type and spa_{KKAA} or spa_{KKAA}/sbi mutant S. aureus. As expected, the staphylococcal load in organ tissues from spa_{KKAA} infected animals was lower than that of mice infected with wild-type S. aureus (FIG. 3C). Mice infected with the spa_{KKAA}/sbi mutant did not display a further reduction in staphylococcal load (FIG. 3C). The virulence defects of wild-type spa were abolished in μMT mice, as similar numbers of bacteria were isolated from organ tissues of animals infected with wild-type, spa_{AA} or spa_{KKAA}/sbi infected mice (FIG. 3C). Further, similar staphylococcal loads were determined in C57BL/6 and μMT mice infected with either spa_{AA} or spa_{KKAA}/sbi mutants (FIG. 3C). These data therefore suggest that natural antibodies do not provide protection and that Sbi binding to IgG does not contribute to S. aureus virulence in C57BL/6 mice. In summary, our results implicate protein A as a key virulence factor that promotes S. aureus escape from opsonophagocytosis by binding IgG Fcγ domains and coating the bacterial surface with immunoglobulin. Protein A crosslinking of B cell receptors prevents the development of protective antibody responses against many different virulence factors, which would otherwise establish immunity and prevent recurrent infections.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Atkins, et al., Mol. Immunol. 45: 1600, 2008.
Baba, et al., J. Bacteriol. 190: 300, 2007.
Bae & Schneewind, Plasmid 55: 58, 2005.
Bhakdi & Tranum-Jensen, Microbiol. Rev. 55: 733, 1991.
Bjerketorp, et al., FEMS Microbiol. Lett. 234: 309, 2004.
Bjerketorp, et al. Microbiology, 148: 2037, 2002.
Bouchard, et al., J. Bacteriol. 194: 6292, 2012.
Bowden, et al., J. Biol. Chem. 283: 2008.
Bubeck-Wardenburg & Schneewind, J. Exp. Med. 205: 287, 2008.
Burts, et al., Proc. Natl. Acad. Sci. USA 102: 1169, 2005.
Butler, et al., Dev. Comp. Immunol. 33: 321, 2009.
Cary, et al., Mol. Immunol. 36: 769, 1999.
Cheng et al., FASEB J. 23: 3393, 2009.
Cheng et al., PLoS Pathog. 6: e1001036, 2010.
Cheng, et al., Trends Microbiol. 19: 225, 2011.
Daum & Spellberg, Clin. Infect. Dis. 54: 560, 2012.
de Haas et al., J. Exp. Med. 199: 687, 2004.
Deisenhofer, et al., Physiol. Chem. 359: 975, 1978.
Deivanayagam, et al., EMBO J. 21: 6660, 2002.
DeLeo, et al., Nat. Rev. Microbiol. 7: 629, 2009.
Enright, et al., J. Clin. Microbiol. 38: 1008, 2000.
Falugi, “Staphylococcus aureus evasion of host adaptive immune responses”, sub. 2013.
Falugi, et al., MBio. 5: e00575, 2013.
Fitzgerald, MBio. 3: e00082, 2012.
Fitzgerald, Trends Microbiol. 20: 192, 2012.
Flick, et al., Blood 10: 1783, 2013.
Fluit, et al., Clin. Microbiol. Infect. 15: 735, 2012.
Forsgren & Sjoquist, J. Immunol. 97: 822, 1966.
Forsgren, et al., Eur. J. Immunol. 6: 207, 1976.
Forsgren, et al., J. Immunol. 112: 1177, 1974.
Forsgren, Infect. Immun. 2: 672, 1970.
Fridkin et al., N. Engl. J. Med. 352: 1436, 2005.
Friedrich, et al., J. Bio. Chem. 281: 1188, 2006.
Friedrich, et al., Nature 425: 535, 2003.
Ganesh, et al., PLoS Pathog. 4: e1000226, 2008.
Goodyear & Silverman, J Exp Med. 197: 1125, 2003.
Graille et al., Proc. Nat. Acad. Sci. USA 97: 5399, 2000.
Hale, et al., Br. J. Exp. Pathol. 26: 209, 1945.
Haupt et al., PloS Pathog. 4: e1000250, 2008.
Hawiger, et al., Biochemistry 21: 1407, 1982.
Kallen et al., JAMA 304: 641, 2010.
Kanemitsu, et al., Micriobiol. Immunol. 45: 23, 2001.
Kennedy et al., Proc. Natl. Acad. Sci. USA 105: 1327, 2008.
Kim et al., Infect. Immun. 80: 3460, 2012.
Kim, et al., Curr. Opin. Microbiol. 15: 92-99, 2012.
Kim, et al., FASEB J. 25: 3605, 2011.
Kim, et al., J. Exp. Med. 207: 1863, 2010.
Klevens et al., JAMA 298: 1763, 2007.
Klevens, et al., Clin. Infect. Dis. 47: 927, 2008.
Kroh, et al., Proc. Natl. Acad. Sci. USA 106: 7786, 2009.
Lindmark, et al., J. Immunol. Methods 62:1, 1983.
Lowy, New Engl. J. Med. 339: 520, 1998.
Mazmanian et al., Science 299: 906, 2003.
Mazmanian, et al., Proc. Natl. Acad. Sci. USA 97: 5510, 2000.
Mazmanian, et al., Proc. Natl. Acad. Sci. USA 99: 2293, 2002.
Mazmanian, et al., Science 285: 760, 1999.
McAdow et al., PLoS Pathog. 7: e1002307, 2011.
McAdow, et al., Infect. Immun. 80: 3389, 2012.
McCarthy, et al., BMC Microbiology 10: 173, 2010.
McDevitt, et al., Eur. J. Biochem. 247: 416, 1997.
McDevitt, et al., Mol. Microbiol. 11: 237, 1994.
McDevitt, et al., Mol. Microbiol. 16: 895, 1995.
Middleton, et al., Vet. Microbiol. 134: 192, 2009.
Moreillon et al., Infect. Immun. 63: 4738, 1995.
Much, Biochem. Z. 14: 143, 1908.
O'Connell, et al., J. Biol. Chem. 273: 6821, 1998.
Palmqvist, et al., Microb. Infect. 6: 188, 2004.
Palmqvist, et al., Microbes Infect. 7: 1501, 2005.
Panizzi, et al., Cell. Mol. Life Sci. 61: 2793, 2004.
Panizzi, et al., J. Biol. Chem. 281: 1179, 2006.
Panizzi, et al., Nat. Med. 17: 1142, 2011.
Patel, et al., J. Immunol. 184: 6283, 2010.
Ponnuraj, et al., Cell 115: 217, 2003.
Price, et al., MBio. 3: e00305, 2012.
Rivas, et al., Curr. Opin. Drug Discov. Devel. 7: 223, 2004.
Rooijakkers et al., Nat. Immunol. 6: 920, 2005.
Sasso, et al., J. Immunol. 142: 2778, 1989.
Schneewind, et al., Science 268: 103, 1995.
Silverman, et al., J. Immunol. 161: 5720, 1998.
Sjodahl, Eur. J. Biochem. 73: 343, 1977.
Smith, et al., J. Clin. Microbiol. 43: 4737, 2005.
Smith, et al., Nature 178: 982, 1956.
Spoor, et al., MBio. 13, 2013.
Streitfeld, et al., Nature 184: 1665, 1959.
Strong, et al., Biochemistry 21: 1414, 1982.
Subedi, et al., Microbiology 153: 3235, 2007.
Thammavongsa, et al., J. Exp. Med. 206: 2417, 2009.
Thomer, et al., J. Bio. Chem. 288: 28283, 2013.
Ton-That, et al., Proc. Natl. Acad. Sci. USA 96: 12424, 1999.
Ulén, et al., J. Biol. Chem. 259: 1695 and 13628, 1984.
Viana, et al., Mol. Microbiol. 77:1583, 2010.
Watanabe, et al., J. Bacteriol. 187: 3698, 2005.
Watanabe, et al., PLOS One 4: e5714, 2009.
Zhang, et al., Microbiology 144: 985, 1998.

1-81. (canceled)

82. An isolated recombinant staphylococcal bacteria that expresses a variant Protein A (SpA) comprising (a) at least one amino acid substitution that disrupts Fc binding and (b) at least a second amino acid substitution that disrupts VH3 binding in at least one of SpA D, A, B, C or E domains.

83. The isolated recombinant staphylococcal bacteria of claim 82, wherein the variant SpA comprises an amino acid substitution in the D, A, B, C and E domain.

84. The isolated recombinant staphylococcal bacteria of claim 82, wherein the variant SpA comprises
an amino acid substitution in the D domain at one or more positions corresponding to position 9, 10, 36 or 37 of SEQ ID NO: 2 and the variant SpA has a D domain that is at least 80% identical to the amino acid sequence of SEQ ID NO: 2,
an amino acid substitution in the A domain at one or more positions corresponding to positions 7, 8, 34 and 35 of SEQ ID NO: 4 and the variant SpA has an A domain that is at least 80% identical to the amino acid sequence of SEQ ID NO: 4,
an amino acid substitution in the B domain at one or more positions corresponding to positions 7, 8, 34, or 35 of SEQ ID NO: 6 and the variant SpA has an B domain that is at least 80% identical to the amino acid sequence of SEQ ID NO: 6,
an amino acid substitution in the C domain at one or more corresponding to positions 7, 8, 34, or 35 of SEQ ID NO: 5 and the variant SpA has an C domain that is at least 80% identical to the amino acid sequence of SEQ ID NO: 5, and/or
an amino acid substitution in the E domain at one or more corresponding to positions 6, 7, 33, or 34, of SEQ ID NO: 3 and the variant SpA has an E domain that is at least 80% identical to the amino acid sequence of SEQ ID NO: 3.

85. The isolated recombinant staphylococcal bacteria of claim 84, wherein the amino acid substitution corresponding to position 9 and 10 of SEQ ID NO: 2, position 7 and 8 of SEQ ID NO: 4, position 7 and 8 of SEQ ID NO: 6, position 7 and 8 of SEQ ID NO: 5, and position 6 and 7 of SEQ ID NO: 3 is a lysine substitution.

86. The isolated recombinant staphylococcal bacteria of claim 85, wherein the amino acid substitution corresponding to position 36 and 37 of SEQ ID NO: 2, position 33 and 34 of SEQ ID NO: 4, position 33 and 34 of SEQ ID NO: 6, position 33 and 34 of SEQ ID NO: 5, and position 33 and 34 of SEQ ID NO: 3 is an alanine substitution.

87. The isolated recombinant staphylococcal bacteria of claim 82, further comprising a second, non-Protein A, antigen segment.

88. The isolated recombinant staphylococcal bacteria of claim 87, wherein the second antigen segment is a staphylococcal antigen segment.

89. The isolated recombinant staphylococcal bacteria of claim 88, wherein the staphylococcal antigen segment is an Emp, EsxA, EsxB, EsaC, Eap, Ebh, EsaB, Coa, vWbp, vWh, Hla, SdrC, SdrD, SdrE, IsdA, IsdB, IsdC, ClfA, ClfB, and/or SasF segment.

90. The isolated recombinant staphylococcal bacteria of claim 82, wherein the bacteria further comprise a heterologous drug susceptibility determinant.

91. The isolated recombinant staphylococcal bacteria of claim 82, wherein the staphylococcal bacterium is S. aureus.

92. A vaccine composition comprising a live attenuated staphylococcal bacteria, wherein the staphylococcal bacteria expresses a variant Protein A (SpA) comprising (a) at least one amino acid substitution that disrupts Fc binding and (b) at least a second amino acid substitution that disrupts VH3 binding in at least one of SpA D, A, B, C or E domains.

93. The vaccine composition of claim 92, wherein the variant SpA comprises an amino acid substitution in the D domain at one or more positions corresponding to position 9, 10, 36 or 37 of SEQ ID NO: 2 and the variant SpA has a D domain that is at least 80% identical to the amino acid sequence of SEQ ID NO: 2,
an amino acid substitution in the A domain at one or more positions corresponding to positions 7, 8, 34 and 35 of SEQ ID NO: 4 and the variant SpA has an A domain that is at least 80% identical to the amino acid sequence of SEQ ID NO: 4,
an amino acid substitution in the B domain at one or more positions corresponding to positions 7, 8, 34, or 35 of SEQ ID NO: 6 and the variant SpA has an B domain that is at least 80% identical to the amino acid sequence of SEQ ID NO: 6,
an amino acid substitution in the C domain at one or more corresponding to positions 7, 8, 34, or 35 of SEQ ID NO: 5 and the variant SpA has an C domain that is at least 80% identical to the amino acid sequence of SEQ ID NO: 5, and/or
an amino acid substitution in the E domain at one or more corresponding to positions 6, 7, 33, or 34, of SEQ ID NO: 3 and the variant SpA has an E domain that is at least 80% identical to the amino acid sequence of SEQ ID NO: 3.

94. The vaccine composition of claim 93, wherein the amino acid substitution corresponding to position 9 and 10 of SEQ ID NO: 2, position 7 and 8 of SEQ ID NO: 4, position 7 and 8 of SEQ ID NO: 6, position 7 and 8 of SEQ ID NO: 5, and position 6 and 7 of SEQ ID NO: 3 is a lysine substitution and the amino acid substitution corresponding to position 36 and 37 of SEQ ID NO: 2, position 33 and 34 of SEQ ID NO: 4, position 33 and 34 of SEQ ID NO: 6, position 33 and 34 of SEQ ID NO: 5, and position 33 and 34 of SEQ ID NO: 3 is an alanine substitution.

95. The vaccine composition of claim 92, wherein the bacterium comprises a heterologous drug susceptibility determinant.

96. The vaccine composition of claim 95, wherein the staphylococcal bacterium is S. aureus.

97. A method for treating a staphylococcal infection in a subject comprising providing to a subject having, suspected of having or at risk of developing a staphylococcal infection an effective amount of a composition comprising a live attenuated staphylococcal bacteria, wherein the staphylococcal bacteria that expresses a variant Protein A (SpA) comprising (a) at least one amino acid substitution that disrupts Fc binding and (b) at least a second amino acid substitution that disrupts VH3 binding in at least one of SpA D, A, B, C or E domains.

98. The method of claim 97, wherein the variant SpA comprises
an amino acid substitution in the D domain at one or more positions corresponding to position 9, 10, 36 or 37 of SEQ ID NO: 2 and the variant SpA has a D domain that is at least 80% identical to the amino acid sequence of SEQ ID NO: 2,
an amino acid substitution in the A domain at one or more positions corresponding to positions 7, 8, 34 and 35 of SEQ ID NO: 4 and the variant SpA has an A domain that is at least 80% identical to the amino acid sequence of SEQ ID NO: 4,
an amino acid substitution in the B domain at one or more positions corresponding to positions 7, 8, 34, or 35 of SEQ ID NO: 6 and the variant SpA has an B domain that is at least 80% identical to the amino acid sequence of SEQ ID NO: 6,
an amino acid substitution in the C domain at one or more corresponding to positions 7, 8, 34, or 35 of SEQ ID NO: 5 and the variant SpA has an C domain that is at least 80% identical to the amino acid sequence of SEQ ID NO: 5, and/or
an amino acid substitution in the E domain at one or more corresponding to positions 6, 7, 33, or 34, of SEQ ID NO: 3 and the variant SpA has an E domain that is at least 80% identical to the amino acid sequence of SEQ ID NO: 3.

99. The method of claim 98, wherein the staphylococcal infection is resistant to one or more treatments.

100. The method of claim 98, wherein the staphylococcal infection is methicillin resistant.

101. The method of claim 98, further comprising administering to the subject a biological response modifier.


SEQUENCE LISTING (13 sequences)

SEQ ID NO: 1 - protein, 291 aa, Staphylococcus sp.
AQHDEAKKNAFYQVLNMPNLNADQRNGFIQSLKAAPSQSANVLGEAQKLNDSQAPKADAQQNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 2 - protein, 54 aa, Staphylococcus sp.
QQNNFNKDQQSAFYEILNMPNLNEAQRNGFIQSLKDDPSQSTNVLGEAKKLNES

SEQ ID NO: 3 - protein, 51 aa, Staphylococcus sp.
QHDEAQQNAFYQVLNMPNLNADQRNGFIQSLKDDPSQSANVLGEAQKLNDS

SEQ ID NO: 4 - protein, 52 aa, Staphylococcus sp.
NNFNKEQQNAFYEILNMPNLNEEQRNGFIQSLKDDPSQSANLLSEAKKLNES

SEQ ID NO: 5 - protein, 52 aa, Staphylococcus sp.
NKFNKEQQNAFYEILHLPNLTEEQRNGFIQSLKDDPSVSKEILAEAKKLNDA

SEQ ID NO: 6 - protein, 52 aa, Staphylococcus sp.
NKFNKEQQNAFYEILHLPNLNEEQRNGFIQSLKDDPSQSANLLAEAKKLNDA

SEQ ID NO: 7 - DNA, 57 bp, artificial sequence
  synthetic primer
ggggaccactttgtacaagaaagctgggtcatttaagaagattgtttcagatttatg

SEQ ID NO: 8 - DNA, 51 bp, artificial sequence
  synthetic primer
atttgtaaagtcatcataatataacgaattatgtattgcaatactaaaatc

SEQ ID NO: 9 - DNA, 46 bp, artificial sequence
  synthetic primer
cgtcgcgaactataataaaaacaaacaatacacaacgatagatatc

SEQ ID NO: 10 - DNA, 56 bp, artificial sequence
  synthetic primer
ggggacaagtttgtacaaaaaagcaggcaacgaacgcctaaagaaattgtctttgc

SEQ ID NO: 11 - DNA, 43 bp, artificial sequence
  synthetic primer
cataattcgttatattatgatgactttacaaatacatacaggg

SEQ ID NO: 12 - DNA, 38 bp, artificial sequence
  synthetic primer
gtattgtttgtttttattatagttcgcgacgacgtcca

SEQ ID NO: 13 - protein, 520 aa, Staphylococcus sp.
MLTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNNFNKDQQSAFYEILNMPNLNEAQRNGFIQSLKDDPSQSTNVLGEAKKLNESQAPKADNNFNKEQQNAFYEILNMPNLNEEQRNGFIQSLKDDPSQSANLLSEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDGNKPGKEDNNKPGKEDNKKPGKEDNNKPGKEDNNKPGKEDGNKPGKEDNKKPGKEDNNKPGKEDGNKPGKEDGNGVHVVKPGDTVNDIAKANGTTADKIAADNKLADKNMIKPGQELVVDKKQPANHADANKAQALPETGEENPFIGTTVFGGLSLALGAALLAGRRREL